(19) United States
(12) Patent Application Publication
(10) Pub. No.: US 2023/0079454 A1
(43) Pub. Date: Mar. 16, 2023

(54) MONITORING VIABILITY OF ORGANS FOR TRANSPLANTATION

(71) Applicant: The General Hospital Corporation, Boston, MA (US)

(72) Inventors: Shannon N. Tessier, Framingham, MA (US); Mustafa Korkut Uygun, Boston, MA (US); Heidi Yeh, Boston, MA (US); Mehmet Toner, Charlestown, MA (US); Reinier De Vries, Boston, MA (US)

(21) Appl. No.: 17/799,164

(22) PCT Filed: Feb. 12, 2021
(86) PCT No.: PCT/US2021/017846

§ 371 Date: Aug. 11, 2022

Related U.S. Application Data
(60) Provisional application No. 63/032,454, filed on May 29, 2020.
(60) Provisional application No. 62/976,591, filed on Feb. 14, 2020.

(51) Int. Cl. G01N 33/569; G01N 33/50

(52) U.S. Cl. CPC G01N 33/56966; G01N 33/502; G01N 2800/245

(57) ABSTRACT

Methods for monitoring the viability of a donor organ before and after transplant based on detection and analysis of whole cells released from the organs.

### CLAIM OF PRIORITY

This application claims the benefit of U.S. Patent Application Ser. No. 62/976,591, filed on Feb. 14, 2020, and 63/032,454, filed on May 29, 2020. The entire contents of the foregoing are hereby incorporated by reference.

### FEDERALLY SPONSORED RESEARCH OR DEVELOPMENT

This invention was made with Government support under Grant Nos. DK114506, DK096075, DK107875, and HL1431149 awarded by the National Institutes of Health. The Government has certain rights in the invention.

### TECHNICAL FIELD

Provided herein are methods for monitoring the viability of an organ before and after transplant based on detection and analysis of whole cells released from the organs.

### BACKGROUND

Solid organ transplantation is the unique solution for end-stage organ failure (Monguió-Tortajada et al. Front. Immunol. 5: 416, (2014)). Liver transplantation, for example, is widely accepted as an effective treatment for a variety of liver diseases including cirrhosis, malignancy, acute failure, and metabolic abnormalities. Not only do transplant recipients survive longer (Feltracco et al. World J. Hepatol. 3, 61-71 (2011)), but quality of life is also substantially improved. For example, kidney transplant recipient's lives are no longer dictated by their next required dialysis treatment and end-stage liver cirrhosis patients are relived from frequent ascites drainages with peritonitis an ever-present life-threatening risk.

The global donor organ shortage indirectly claims hundreds of thousands of lives each year in the US alone³. There is a large heterogeneous pool of donor livers of uncertain quality, which, if tapped, could dramatically reduce the gap between supply and demand⁴. However, the use of those organs incurs an increased risk of negative transplant outcomes. Hence the selection of low-risk donor livers to avoid transplant failures and more importantly, the lack of technology to accurately assess graft viability, have precluded the use of a substantial pool of potentially viable donor livers every year.

A transplanted organ suffers from both acute and chronic immune rejection insults which can be defined according to their underlying mechanism and timing of symptoms (Moreau et al., Cold Spring Harb. Perspect. Med. 3, (2013)). To prevent rejection and consequent death or re-transplantation, patients are under constant burden of immunosuppressive regimes, facing opportunistic infections and de novo malignancies (Gutierrez-Dalmau & Campistol, Drugs 67, 1167-1198 (2007)). However, monitoring of graft rejection and titering of immunosuppressants still relies mainly on clinical judgment¹ with absolute confirmation performed with invasive biopsies after organ damage has already occurred. Importantly, it is general accepted that organ injury can be minimized by the timely detection of both acute (Nasr et al., Expert Rev. Mol. Diagn. 16, 1121-1132 (2016)) and chronic rejection (Flechner, Transpl. Int. Off. J. Eur. Soc. Organ Transplant. 29, 20-22 (2016); Khorsandi & Heaton, Transl. Gastroenterol. Hepatol. 1, 25 (2016); Demetris et al. Am. J. Transplant. Off. J. Am. Soc. Transplant. Am. Soc. Transpl. Surg. 16, 2816-2835 (2016); Bhatti & Usman, Cureus 7, e376 (2015); Kloc & Ghobrial, Burns Trauma 2, 3-10 (2014)) events in their early stage, resulting in longer overall graft survival. Thus, the inability to accurately diagnose and manage treatment for rejection is unacceptable, especially since the world is facing a serious donor shortage crisis whereby transplants are meeting less than 10% of global demand (Lewis et al., Cryobiology 72, 169-182 (2016)).

### SUMMARY

Provided herein are methods of determining viability of an organ for transplantation during or after Machine Perfusion (MP), e.g., Sub-Normothermic MP (SNMP) or preservation, e.g., Hypothermic Preservation (HP), the method comprising: providing a sample of perfusate from the MP or preservation storage media; detecting whole cells from the organ in the sample, and determining the identity, level, activity (e.g., activated versus non-activated immune cells) and/or status (e.g., injured versus healthy), of selected cell types in the sample.

The methods can also include comparing the level of the cells of the selected type from the organ to a reference identity, level, activity and/or status of cells of the selected type, wherein the reference level represents a level, activity and/or status in a viable organ; and identifying an organ that has a level, activity and/or status of cells of the selected type that differs from (e.g., differs significantly from) the reference identity, level, activity and/or status as unsuitable for transplant; or identifying an organ that has a level, activity and/or status of cells of the selected type comparable to (e.g., not significantly different from) the reference identity, level, activity and/or status as suitable for transplant.

In some embodiments, once an organ has been identified as having a level of cells of the selected type below the reference level as suitable for transplant, the method further comprises preparing the organ for transplant and optionally transplanting the organ into a suitable recipient.

In some embodiments, the methods further include enriching the sample for cells from the organ.

In some embodiments, the organ is a liver and the cells are hepatocytes (HC), liver sinusoidal endothelial cells (LSEC), Kupffer cells (KC), Pit Cells, hepatic stellate cells (SC), fibroblasts, and/or dendritic cells (DC).

In some embodiments, the organ is a kidney and the cells are endothelial cells, mesangial cells, podocytes, renal pariental cells, juxtaglomerulal cells, proximal tubule cells, thick ascending limb cells, and distal tubule cells.

In some embodiments, the organ is a heart and the cells are coronary endothelial cells, cardiomyocytes, cardiac pacemaker cells, and/or cardiac fibroblasts.

In some embodiments, the organ is a lung and the cells are, pneumocytes, alveolar macrophages, pulmonary dendritic cells, pulmonary fibroblasts, and/or pulmonary endothelial cells.

In some embodiments, the organ is a pancreas and the cells are alpha cells, beta cells, delta cells, pancreatic islet endothelial cells, pancreatic polypeptide cells, acinar cells, and/or pancreatic fibroblasts.

In some embodiments, the organ is a vascularized composite allograft and the cells are rhabdomyocytes, Langerhans cells, keratinocytes, dermal dendritic cells, mast cells, lymphocytes, capillary endothelial cells, Schwann cells, osteoblasts, and/or osteoclasts

In some embodiments, the organ is a small intestine and the cells are Paneth cells, Goblet cells, enteroendocrine cells, enterocytes, intestinal dendritic cells, intestinal macrophages and/or gut endothelial cells.

In some embodiments, determining the identity and level of selected cell types in the sample comprises contacting the sample with antibodies that bind to identifying cell surface antigens and or intracellular proteins on/in the selected cell types, and quantifying cells that are bound to the antibodies.

In some embodiments, determining the identity, level, activity, and/or status of selected cell types in the sample comprises quantification of DNA, RNA, protein, metabolites, methylation status (or other signature post-translational modifications) or other biomolecule indicating identity, level, activity and/or status of the cells.

Also provided herein are methods for detecting donor organ rejection or injury or risk of donor organ rejection or injury in a subject. The methods include providing a sample comprising peripheral blood from the subject; enriching the sample for donor organ-derived cells; and detecting whole cells from the organ in the sample, and determining the identity, level, activity and/or status of selected cell types in the sample.

In some embodiments, the methods include comparing the identity, level, activity and/or status of the cells of the selected type from the organ to a reference identity, level, activity and/or status of cells of the selected type; and identifying a subject who has a level of cells of the selected type above the reference level as having donor organ rejection or at risk of developing donor organ rejection or injury, e.g., within a selected time period, and optionally administering a treatment for rejection or injury or to reduce the risk of rejection or injury to the organ to the subject; or identifying a subject who has a level of cells of the selected type below the reference level as not having donor organ rejection or injury or not at risk of donor organ rejection or injury, e.g., within a selected time period.

In some embodiments, the methods include enriching the sample for cells from the organ. In some embodiments, enriching the sample for donor organ-derived cells comprises separating nucleated cells from other blood components; collecting and removing peripheral leukocytes; contacting the sample with antibodies that bind to identifying cell surface antigens or intracellular proteins in the selected cell types, and quantifying cells that are bound to the antibodies. In some embodiments, the methods include using a microfluidic device for enriching the sample for donor organ-derived cells.

In some embodiments, determining the identity, level, activity and/or status of selected cell types in the sample comprises contacting the sample with antibodies that bind to identifying cell surface antigens on or intracellular proteins in the selected cell types, and quantifying cells that are bound to the antibodies.

In some embodiments, wherein determining the identity, level, activity and/or status of selected cell types in the sample comprises quantification of DNA, RNA, protein, or other biomolecule indicating identity, level, activity and/or status of the cells.

In some embodiments, the organ is a liver and the cells are hepatocytes (HC), liver sinusoidal endothelial cells (LSEC), Kupffer cells (KC), hepatic stellate cells (SC), Pit cells, fibroblasts, and/or dendritic cells (DC).

In some embodiments, the organ is a kidney and the cells are endothelial cells, mesangial cells, podocytes, renal pariental cells, juxtaglomerulal cells, proximal tubule cells, thick ascending limb cells, and distal tubule cells.

In some embodiments, the organ is a heart and the cells are coronary endothelial cells, cariomyocytes, cardiac pacemaker cells, and/or cardiac fibroblasts.

In some embodiments, the organ is a lung and the cells are pneumocytes, alveolar macrophages, pulmonary dendritic cells, pulmonary fibroblasts, and/or pulmonary endothelial cells.

In some embodiments, the organ is a pancreas and the cells are alpha cells, beta cells, delta cells, pancreatic islet endothelial cells, pancreatic polypeptide cells, acinar cells, and/or pancreatic fibroblasts.

In some embodiments, the organ is a vascularized composite allograft and the cells are rhabdomyocytes, Langerhans cells, keratinocytes, dermal dendritic cells, mast cells, lymphocytes, capillary endothelial cells, Schwann cells, osteoblasts, and/or osteoclasts.

In some embodiments, the organ is a small intestine and the cells are Paneth cells, Goblet cells, enteroendocrine cells, enterocytes, intestinal dendritic cells, intestinal macrophages and/or gut endothelial cells.

In some embodiments, determining the identity, level, activity and/or status of selected cell types in the sample comprises (optionally amplifying) and quantifying one or more cell-type specific transcripts or proteins from donor organ-derived cells, wherein the quantity of the cell-type specific transcripts or proteins indicates the identity, level, activity and/or status of donor organ-derived cells.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C: Experimental design and total number of released cells in the perfusates. A. Schematic representation of the research design. The perfusates of fresh (n=4), 24-h-cold ischemic (CI) (n=5) and 72-h-CI (n=4) rat livers that recirculated for 3 h during subnormothermic machine perfusion was collected and analyzed using multi-channel imaging flow cytometry. Fresh fractions of the perfusate that flushed through the liver at the start and end of perfusion were collected and analyzed separately. The experimental CI groups correspond to the previously established CI limit for 100% transplant survival (24-h-CI) and 0% transplant survival (72-h-CI) in rats after SNMP^{20,21}. B. Total number of cells released during perfusion. C. Total number of cells released in the flushes at the start (dark gray) and end (light gray) of perfusion. Error bars: SEM. Stars denote statistical significance (repeated measures two-way ANOVA, followed by Tukey's post-hoc test): *0.01<p<0.05.

FIGS. 2A-C: Release of structural liver cells into the perfusate after cold ischemia. A. Percentages of presumed hepatocytes (top left), sinusoidal endothelial cells (top right), and stellate cells (bottom) in the perfusate, relative to the total number of nucleated cells (TNCs) in the perfusate. The perfusate recirculated during 3 hours of subnormothermic machine perfusion. Stars denote statistical significance (two-way ANOVA, followed by Tukey's post-hoc test): *0.01<p<0.05; **0.001<p<0.01; ***0.0001<p<0.001; ****p<0.0001. Error bars: SEM. B. Imaging flow cytometry for quantification of presumed hepatocytes (CD45−/SE1−/ASGR1+/OX62−), liver sinusoidal endothelial cells (LSECs) (CD45−/SE1+/ASGR1−/OX62−), and stellate cells (CD45−/CD105+/SE1−/CD14+). Left: fresh livers. Right: 24-h-CI livers. C. Representative images of surface marker expression of hepatocytes (top), LSEC (middle), and stellate cells (below). Scale bars: 5 μm.

FIGS. 3A-C: Alterations in liver-resident immune cell release into the perfusate after cold ischemia. A. Percentage of presumed Kupffer cells (left), liver-resident natural killer cells (also known as pit cells) (middle), and dendritic cells (right) in the perfusate, relative to the total number of nucleated cells (TNCs) that are released into the perfusate from fresh (n=4), 24-h-cold ischemic (CI) (n=5), and 72-h-CI (n=4) livers. The perfusate recirculated during 3 hours of subnormothermic machine perfusion. Stars denote statistical significance (two-way ANOVA, followed by Tukey's post-hoc test): *0.01<p<0.05; **0.001<p<0.01; ***0.0001<p<0.001; ****p<0.0001. Error bars: SEM. B. Imaging flow cytometry for the quantification of presumed Kupffer cells (CD45+/CD105−/SE1−/CD14+) and pit cells (CD45+/NKRP1A+/CD3−/OX62−). Dendritic cells were manually selected from a CD45+/NKRP1A−/CD3−/OX62+ population (not shown). Left: fresh livers. Right: 24-h-CI livers. C. Representative images of surface marker expression images of Kupffer cells (top), pit cells (middle), and dendritic cells (below). Scale bars: 5 μm.

FIGS. 4A-F: Differences in cell release between perfusates collected at the start and end of perfusion. Percentages of liver specific cells released into fresh fractions of perfusate that flushed once through the liver at the start (left hand bars, darker grey) and end (right hand bars, lighter grey) of perfusion. These fractions were collected separately from the perfusate that recirculated during 3 hours of subnormothermic machine perfusion and were analyzed to study the change in cell release. A. Presumed hepatocytes. B. Presumed sinusoidal endothelial cells. C. Presumed stellate cells. D. Presumed Kupffer cells. E. Presumed pit cells. F. Presumed dendritic cells. Percentages are relative to the total number of nucleated cells (TNCs). Stars denote statistical significance (repeated measures two-way ANOVA, followed by Tukey's post-hoc test): *0.01<p<0.05; **0.001<p<0.01; ***0.0001<p<0.001; ****p<0.0001). Error bars: SEM.

FIGS. 5A-E: Correlations between structural liver cell release and corresponding viability parameters. Black: fresh livers (n=4). Medium grey: 24-h-cold ischemic (CI) livers (n=5). Light Grey: 72-h-CI livers (n=4). Black lines: fits of linear regression with 1, 11 degrees of freedom (DFn, DFd). Cell types are expressed as a percentage of the total number nucleated cells (TNCs) released into the perfusate. A. Total bile production vs. percentage of presumed hepatocytes released into the perfusate that recirculated during 3 h of subnormothermic machine perfusion (SNMP). B. Total oxygen uptake (area under the curve) vs. percentage of presumed hepatocytes released into the perfusate during 3 h of SNMP. C. Mean aspartate aminotransferase (AST) concentration in the hepatic vein vs. the percentage of presumed hepatocytes released into the perfusate during 3 h of SNMP. D. Vascular resistance at the start of perfusion vs. the percentage of presumed liver sinusoidal endothelial cell (LSECs) released in the perfusate that flushed once through the liver at the start of perfusion. E. Vascular resistance at the start of perfusion vs. the percentage of presumed stellate cell released at the start of perfusion.

FIGS. 6A-H: Parameters of liver function and injury during subnormothermic machine perfusion. Black circles: fresh livers (n=4). Medium grey squares: 24-h-cold ischemic (CI) livers (n=5). Light Grey diamonds: 72-h-CI livers (n=4). A. Liver weight directly after procurement, before and after perfusion (fresh, pre, and post, respectively) B. Vascular resistance of the portal vein. C. Perfusion pressure in the portal vein. D. Portal flow. E. Cumulative bile production. F. Oxygen uptake calculated from the partial oxygen pressure in the portal and hepatic veins. G. Aspartate aminotransferase (AST) concentration in the hepatic vein. H. Potassium concentration in hepatic vein. Note that the initial drop in AST and potassium between t=0 and 30 min is due to the nature of the experimental design whereby the first fraction of perfusate was removed for imaging flow cytometry. Letters and star denote statistical significance (repeated measures two-way ANOVA, followed by Tukey's post-hoc test; p<0.05); a: fresh vs. 24-h-CI; b: fresh vs. 72-h-CI; c: 24-h-CI vs. 72-h-CI.

FIGS. 7A-C: Energy status of liver tissue after cold ischemia and subsequent subnormothermic machine perfusion. Black: fresh livers (n=4). Medium grey: 24-h-cold ischemic (CI) livers (n=5). Light Grey: 72-h-CI livers (n=4). The energy status was measured in biopsies taken directly after sub normothermic machine perfusion. A. Ratios between adenosine triphosphate (ATP) and adenosine monophosphate (AMP). B. Ratios between ATP and adenosine biphosphate (ADP). C. Adenylate energy charged, defined by the ratio between (ATP+0.5*ADP) and (ATP+ADP+AMP). Stars denote statistical significance (repeated measures two-way ANOVA, followed by Tukey's post-hoc test): *0.01<p<0.05; **0.001<p<0.01. Error bars: SEM.

FIGS. 8A-B: Morphological analysis after cold ischemia and subsequent subnormothermic machine perfusion. Light microscopy images of parenchymal liver biopsies stained for hematoxylin and eosin staining taken directly after perfusion. A. Representative images for 24-h-CI livers. Left: well preserved lobular architecture with mostly patent sinusoids and conspicuous LSEC. Right: Focal mild congestion of liver sinusoids. Arrow 1: focal disruption of the endothelial lining with detached cells and cellular debris in the lumen. B. Representative images for 72-h-CI livers. Arrow 2: Focal disruption of endothelial lining with detached cells, eosinophilic granular and cellular debris in the central vein lumen. Arrows 3: LSECs appear very loosely attached and displaced by perisinusoidal subendothelial edema. Scale bars: 100 μm.

FIGS. 9A-B. Exemplary schematic of pre- and post-transplant assessment methods. A. pre-transplant assessment of organ viability to determine fitness for transplant. B. post-transplant assessment of organs to detect acute and chronic rejection.

FIG. 10. Schematic of iChip illustrating the 3 steps: (1) hydrodynamic sorting of nucleated cells (or microfluidic debulking), (2) inertial focusing within a single file and (3) magnetic sorting of tagged cells.

### DETAILED DESCRIPTION

The current lack of quantitative, objective, and graft-specific diagnostics to effectively evaluate graft viability and accurately predict transplant outcome has critically limited the availability of organs such as livers and kidneys for transplantation. Instead, current clinical standards use only gross population-based donor risk statistics (such as age, race, height, among others)⁵′6. Because of such non-specific indicators coupled with the unacceptable costs of an unsuccessful transplant, perfectly good donor organs are discarded every year. Identifying novel biomarkers that can predict safe utilization of the grafts that are currently discarded would significantly help relieve the donor organ shortage.

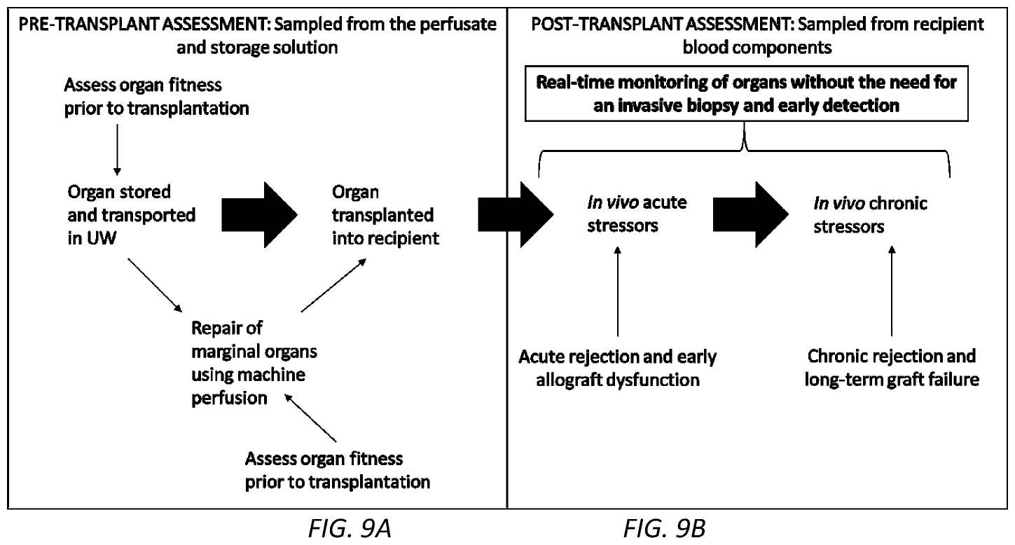

Provided herein are methods for assessing viability to determine fitness for transplantation of donor organs (FIG. 9A).

In addition, acute allograft rejection remains a prevalent and serious problem in transplantation, with an incidence of up to 30-40% in liver (Ziolkowski et al. Transplant. Proc. 35, 2289-2291 (2003)), lung (Martinu et al., Clin. Chest Med. 32, 295-310 (2011)), kidney (Hamida et al., Saudi J. Kidney Dis. Transplant. Off. Publ. Saudi Cent. Organ Transplant. Saudi Arab. 20, 370-374 (2009)), and heart (Hertz et al., J. Heart Lung Transplant. Off. Publ. Int. Soc. Heart Transplant. 28, 989-992 (2009)) transplantation. Although an acute rejection episode is rarely fatal, these episodes lead to organ injury, compromise the patient's immune system, and are major determinants of long-term allograft survival (Hamida et al. (2009), supra; Jalalzadeh et al., Nephro-Urol. Mon. 7, (2015)). In fact, acute rejection events decreases allograft half-life by 34% and increases the risk of chronic rejection (Ingulli, Pediatr. Nephrol. Berl. Ger. 25, 61-74 (2010)) with inherent increased mortality. Occurring between 1 week to several months after transplantation (Moreau et al., Cold Spring Harb. Perspect. Med. 3(11):a015461, (2013)), acute rejection episodes are caused by an immune response directed against the graft, although the mechanism of action can differ substantially. Acute cellular rejection (ACR) is mediated by a T-cell dependent process, while acute antibody-mediated rejection (ABMR; aka “humoral rejection”) is mediated by antibody production against alloantigens (B-cell dependent) (Moreau et al., Cold Spring Harb. Perspect. Med. 3(11):a015461, (2013)). Since cellular versus humoral rejection are mediated by different underlying mechanisms, treatment modalities for each can vary dramatically. Currently, T cell mediated injury uses multimodal application of immunosuppressive agents such as oral or intravenous corticosteroids, antithymocyte globulin, and the murine monoclonal antibody OKT3, which are additional to the preventive regiment of immunosuppressive agents such as ciclosporin, tacrolimus, sirolimus, mycophenolate, or azathioprine. On the other hand, antibody-mediated rejection is relatively less well understood with treatment regimens ranging from IVIG (intravenous immunoglobulin), plasmapheresis, anti-CD20 antibodies (Rituximab), lymphocyte-depleting antibodies (alone or in combination) (Djamali et al., Am. J. Transplant. 14, 255-271 (2014)), as well as corticosteroids. Despite such different underlying mechanisms and treatment courses, distinguishing ACR from ABMR is very challenging and requires invasive biopsies for histological assessment in already sick patients. Yet the impact of not addressing these different disease etiologies can be enormous.

Provided herein are methods that allow the identification of subjects who are rejecting, or are at risk of, rejection, e.g., acute or chronic rejection, of a transplanted organ (FIG. 9B).

In general, each method includes obtaining a sample comprising perfusate or cold storage solution (for pre-transplant assessment) or peripheral blood (for post-transplant assessment); isolating cells from the sample that are from the donor organ; and analyzing the cells. The presence and/or level of cells indicates whether the organ is viable and/or whether the subject is undergoing a rejection process.

The methods described herein are applicable to a wide range of solid organ transplantation, including hearts, livers, kidneys, pancreases, lungs, intestines, and vascularized composite allografts (VCAs).

Monitoring Viability before Transplant: Machine Perfusion (MP) and Hypothermic Preservation (HP)

Advances in machine perfusion have offered several solutions to overcome the organ shortage, including ex vivo viability assessment of marginal donor organs. However, clinical accurate assessment of liver viability during machine perfusion is elusive and would benefit from additional viability biomarkers⁷⁻⁹. Further, fundamentally different ex vivo machine perfusion modalities aimed at resuscitating marginal organs have emerged with unique pros and cons¹⁰⁻¹¹ and understanding of the specific injury mechanisms of each cell type may help improve the different machine perfusion and preservation technologies.

As shown herein, organ type-specific cells are released during graft handling and preservation and can be used for assessing the fitness of an organ prior to transplantation. To the present inventors' knowledge, whole cell release from organs in response to injury and its implications on graft viability have not been studied before. Organ-specific cells could be promising biomarkers because: 1) they can be sampled non-invasively; 2) unlike biopsy tissues, these cells represent the whole organ and capture spatial differences in tissue injury; 3) they can be easily obtained from the flush after hypothermic preservation or from the perfusate during machine perfusion; 4) unlike other blood-based biologics such as cell-free DNA, microparticles, and/or exosomes, these cells are not abundantly shed from normal tissues and can thus be used to specifically identify injury-derived expression signatures; 5) the functional specificity of each cell type could be leveraged to identify and understand complex injury mechanisms.

Thus the methods can include determining organ viability at one or more time points before, during, and/or after machine perfusion. In some embodiments, the perfusion temperature is between 35-38° C., 25-34° C., 13-24° C., 0-12° C. In some embodiments, the MP is Sub-Normothermic Machine Perfusion (SNMP). NMP; normothermic machine perfusion (35° C.-38° C.); SNMP, subnormothermic machine perfusion (25° C.-34° C.); MMP, mid-thermic machine perfusion (13° C.-24° C.); HMP, hypothermic machine perfusion (0° C.-12° C.). See, e.g. Karangwa et al., Am J Transplant. 2016 October; 16(10): 2932-2942.

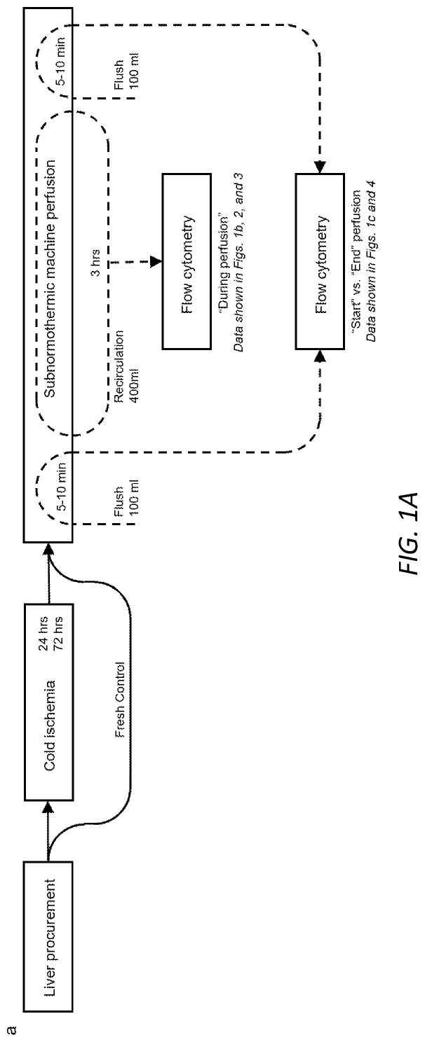

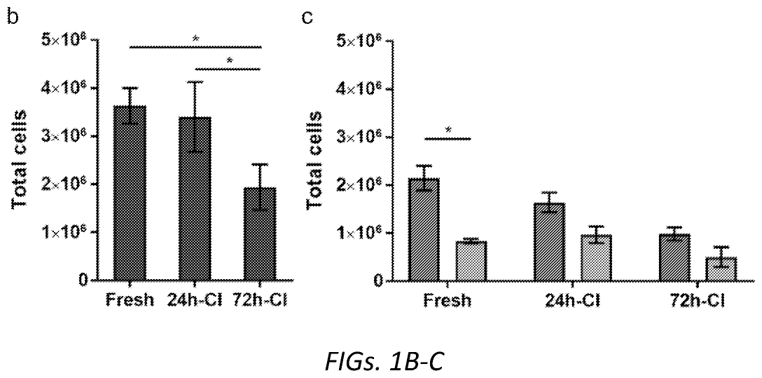

A concern was that there might not be enough cells in machine perfusate. Instead we found that millions of cells were released during perfusion of livers (FIGS. 1A-C). In this study, we processed the complete perfusate volume for imaging flow cytometry analysis. Based on the result that 400 ml of perfusate contained approximately 4 M cells (FIG. 1A-C), a 2 ml perfusate sample would provide 20,000 cells, which is sufficient for imaging flow cytometry analysis. Moreover, the perfusate-to-tissue volume ratios were approximately 50 times larger in human liver perfusion compared with rat liver perfusion (1:1 vs. 1:50 gₜᵢₛₛᵤₑ/ml perfusate, respectively), which suggests that sample volumes as little as 50 μl can be used for analysis. Microfluidic chip technology could likely provide real-time measurement in just a droplet of perfusate.

Thus, the present methods can include obtaining a sample of perfusate at one or more time points during machine perfusion of an organ, and detecting the presence, identity, level, activity and/or status of cells in the sample.

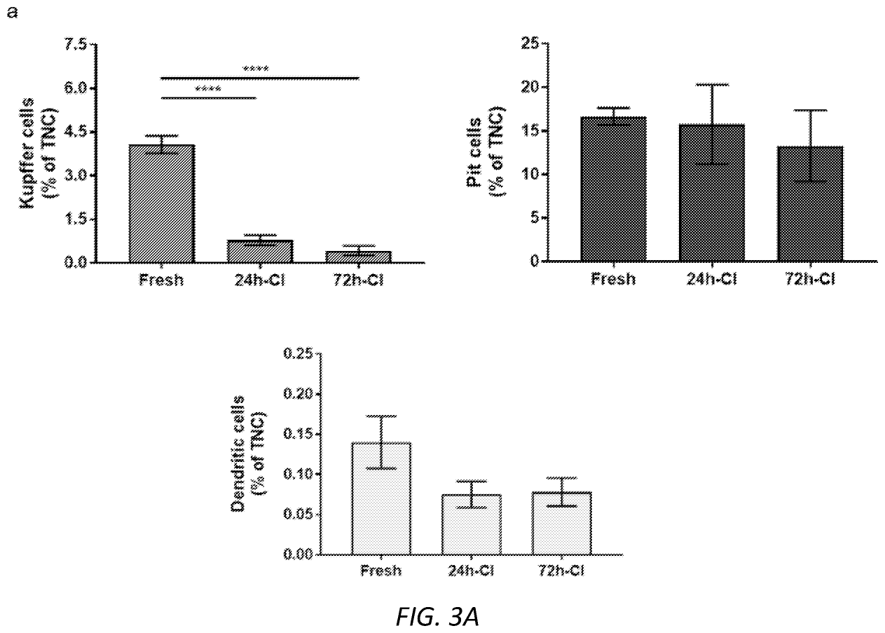

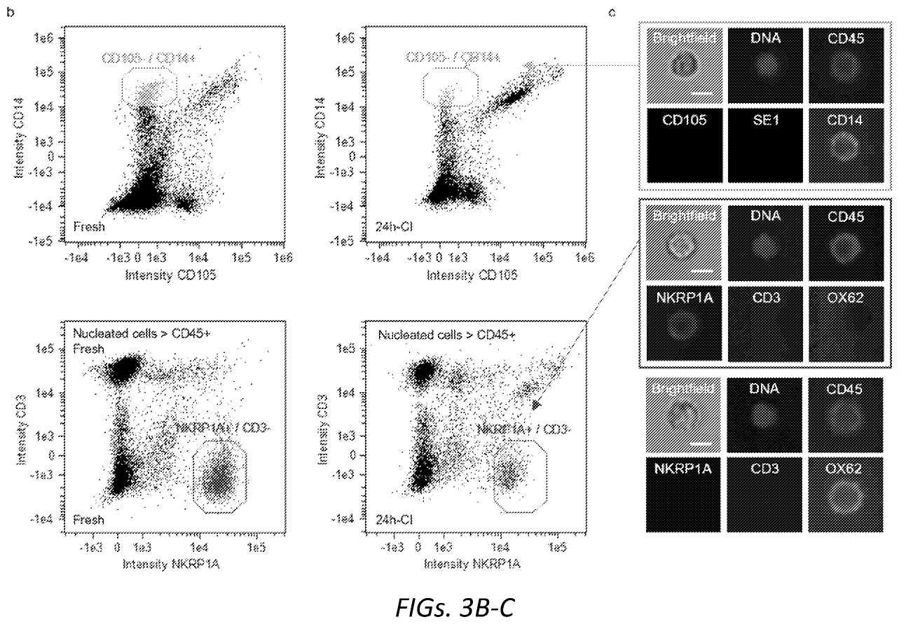

Although machine perfusion is an emerging preservation technology, many transplanted livers are not machine perfused, hypothermic preservation (HP) is still the current clinical standard. Cells released in the HP storage solution and can be analyzed in the present methods without the need of machine perfusion. In this regard, in livers we found that hepatocytes^{p}, LSECs^{p} and Kupffer^{p} cells (FIG. 3) were released at the very start of perfusion and that this markedly changed as result of cold ischemia. This very early release of cells during perfusion suggest that these cells were already released in the HP solution.

Thus, the present methods can include obtaining a sample of storage solution at one or more time points during preservation of an organ, and detecting the identity, level, activity and/or status of cells in the sample.

In some embodiments, the organ is a liver and the cells are hepatocytes (HC), liver sinusoidal endothelial cells (LSEC), Kupffer cells (KC), Pit Cells, hepatic stellate cells (SC), fibroblasts, and/or dendritic cells (DC).

In some embodiments, the organ is a kidney and the cells are endothelial cells, mesangial cells, podocytes, renal pariental cells, juxtaglomerulal cells, proximal tubule cells, thick ascending limb cells, and distal tubule cells.

In some embodiments, the organ is a heart and the cells are coronary endothelial cells, cardiomyocytes, cardiac pacemaker cells, and/or cardiac fibroblasts.

In some embodiments, the organ is a lung and the cells are, pneumocytes, alveolar macrophages, pulmonary dendritic cells, pulmonary fibroblasts, and/or pulmonary endothelial cells.

In some embodiments, the organ is a pancreas and the cells are alpha cells, beta cells, delta cells, pancreatic islet endothelial cells, pancreatic polypeptide cells, acinar cells, and/or pancreatic fibroblasts.

In some embodiments, the organ is a vascularized composite allograft and the cells are rhabdomyocytes, Langerhans cells, keratinocytes, dermal dendritic cells, mast cells, lymphocytes, capillary endothelial cells, Schwann cells, osteoblasts, and/or osteoclasts

In some embodiments, the organ is a small intestine and the cells are Paneth cells, Goblet cells, enteroendocrine cells, enterocytes, intestinal dendritic cells, intestinal macrophages and/or gut endothelial cells.

The identity, activity and/or status of a cell can be determined based on, e.g., the presence or absence of cell type-specific biomolecules, e.g., proteins or transcripts; the level of a cell type can be determined by quantifying cells directly or indirectly based on cell type-specific biomolecules.

For example, for Kupffer cells (a type of liver-specific immune cells) CD14 antigen expression on the cell membrane goes up if the Kupfer cells are activated. Other Kupffer cell activation markers include CD163 and CD33. Activated vs non-activated Kupfer cells can also be distinguished based on the intracellular RNA and protein expression of specific cytockines such as IL-12 and IL-23, among others.

For injury to cells (for example to hepatocytes) markers of cell death such as apoptosis or necrosis markers can be used to determine status. General markers of impending cell death include, but are not limited to, loss of membrane symmetry, intracellular caspase activation, cytochrome-c release from the mitochondria, nuclear condensation, and DNA fragmentation.

The methods can include sorting cells based on identity and determining quantity, status, and activity.

In some embodiments, the identity, level, activity and/or status of a donor organ-derived cell is different from, e.g., significantly different from, (either above or below, depending on the cell type as described herein) comparable to the identity, level, activity and/or status of the cells(s) in a reference organ that is viable and/or suitable for transplantation, then the donor organ is deemed unsuitable for transplantation.

Suitable reference values can be determined using methods known in the art, e.g., using standard clinical trial methodology and statistical analysis. The reference values can have any relevant form. In some cases, the reference comprises a predetermined value for a meaningful identity, level, activity and/or status of a marker, e.g., a control reference identity, level, activity and/or status that represents a normal identity, level, activity and/or status of the marker, e.g., an identity, level, activity and/or status in a organ (or cohort of organs) that are suitable for transplantation, and/or disease reference that represents an identity, level, activity and/or status of the cells associated with non-viable grafts.

The predetermined identity, level, activity and/or status can be a single cut-off (threshold) value, such as a median or mean, or an identity, level, activity and/or status that defines the boundaries of an upper or lower quartile, tertile, or other segment of a set of donor organs that is determined to be statistically different from the other segments. It can be a range of cut-off (or threshold) values, such as a confidence interval. It can be established based upon comparative groups, such as where association with viability or non-viability in one defined group is a fold higher, or lower, (e.g., approximately 2-fold, 4-fold, 8-fold, 16-fold or more) than the viability or non-viability in another defined group. It can be a range, for example, where a set of organs is divided equally (or unequally) into groups, such as a non-viable group, a medium-viability group and a high viability group, or into quartiles, the lowest quartile being organs with the lowest viability score and the highest quartile being organs with the highest viability, or into n-quantiles (i.e., n regularly spaced intervals) the lowest of the n-quantiles being subjects with the lowest viability and the highest of the n-quantiles being subjects with the highest viability.

In some embodiments, the predetermined identity, level, activity and/or status is an identity, level, activity and/or status or occurrence in the same organ, e.g., at a different time point, e.g., an earlier time point.

Organs or cohorts of organs associated with predetermined values are typically referred to as reference organs.

In characterizing likelihood, or risk, numerous predetermined values can be established.

The present methods can include calculating composite cell indices or scores using a combination of donor organ-derived cell types to account for differences in susceptibilities and different injury mechanisms. For example, in livers sinusoidal endothelial cells have been shown to be more susceptible to cold ischemia, whereas warm ischemia seems more injurious to Kupfer cells. A composite index may therefore more accurately capture the multimodal injury mechanisms in human liver transplantation in future clinical studies. Thus an algorithm, e.g., a linear algorithm, e.g., wherein identity, level, activity and/or status of a plurality of cell types can be used to calculate a composite score that represents the viability of the organ. In some embodiments, the values for each cell type (optionally weighted to increase or decrease its effect on the final score) can be summed to produce a viability score.

Thus, the present methods can include obtaining a sample of HP solution at one or more time points during preservation of an organ, and detecting the identity, level, activity and/or status of cells in the sample. If an organ is deemed to be viable, the methods can include transplanting the organ into a suitable recipient. Methods for identifying suitable recipients are known in the art.

Monitoring Viability after Transplant Organ Rejection and Injury

A record breaking 34,768 transplants were performed in the USA in 2017, and these numbers are expected to grow in subsequent years. While a lifesaving procedure, once an organ is transplanted recipients are under the constant fear of rejection and are sentenced to a lifetime of immunosuppressive therapy. Despite this reality, the availability of clinical assays to accurately quantify and assess the immunosuppression state as well as diagnose acute or chronic organ rejection or injury, especially in the early stages, is severely lacking. This is critical since the field of transplantation is facing a critical donor shortage crisis whereby graft failure can mean death for many patients who won't be lucky enough to get a second transplant. Thus, monitoring based solely on clinical judgement and titering of immunosuppressants (Germani et al. World J. Gastroenterol. WJG 21, 1061-1068 (2015)) is simply no longer acceptable and the field is currently facing significant unmet needs. More specifically, the field is facing three critical bottlenecks: 1) assays which show high specificity yet require highly invasive tissue biopsies, 2) assessment of biomarkers which overlap with other disease etiologies, or 3) can only detect late stages of rejection once organ injury has already occurred. The present methods include the use of a non-invasive liquid biopsy that is amenable to continuous monitoring, predictive of tolerance development, yet also offers improved sensitivity and specificity.

The present methods can also be used to non-invasively monitor viability of a transplanted organ after transplant into a recipient, e.g., to measure the immunosuppressive state of the graft itself and identify subjects who are developing or are at risk of acute or chronic rejection or injury to the organ. These methods include detection of rare circulating cells derived from the recipient graft itself using microfluidics. Vascularized organs shed passenger cells from the transplanted organ which migrate through the recipients circulatory system (Rutkowska et al. Ann. Transplant. 12, 12-14 (2007); Rao et al., Curr. Opin. Nephrol. Hypertens. 3, 589-595 (1994)).

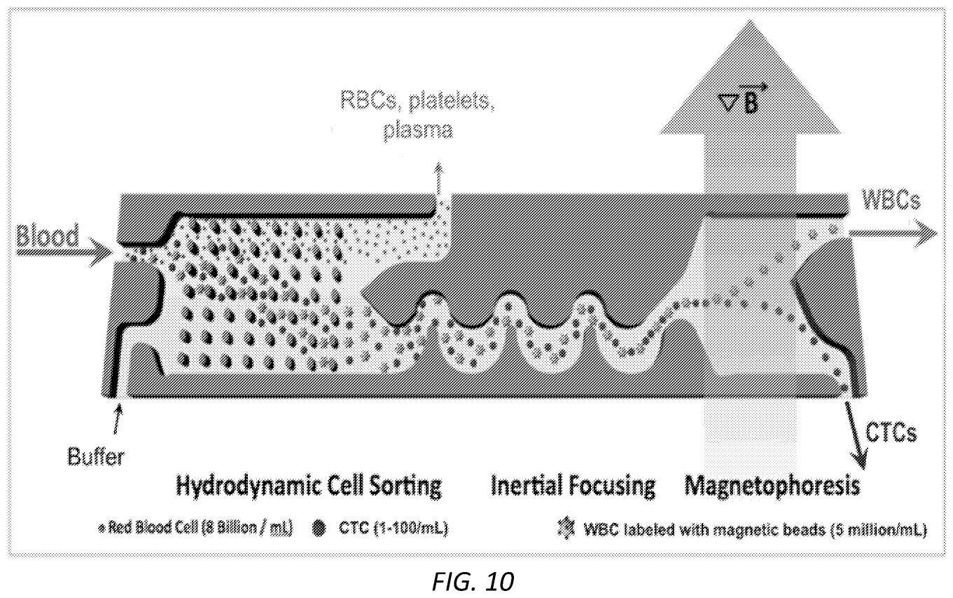

Despite the potential of leveraging donor passenger cells shed into the recipient's bloodstream, these cells have not been used to monitor transplant patients, possibly due to technical barriers which preclude downstream analysis. Others have shown that 0.7-5.1 circulating hepatocytes per mL are present in peripheral blood collected from patients with chronic liver disease. With ˜5 million cells per mL of blood and taking the upper limit of circulating hepatocytes (i.e. 5 cells/mL), this results in an abundance of ˜1 target cell/million. Thus, the present disclosure makes use of sorting technologies to overcome these obstacles. A recently introduced microfluidic device, called the iChip (Ozkumur et al. Sci Transl Med. 2013; 5(179):179ra47; Karabacak et al. Nat Protoc. 2014; 9(3):694-710; Kalinich et al. Proc Natl Acad Sci USA. 2017; 114(5):1123-1128) can be used to isolate one circulating tumor cell from 5 mL of blood with a 97% efficiency (Karabacak et al. Nat. Protoc. 9, 694-710 (2014)). The iChip is based on the principle of depleting normal blood cells away from untagged rare cells (termed “negative depletion”). With this cell sorting technology, whole blood is processed at a rate of 30 million cells per second. See FIG. 10. Other methods can also be used, e.g., as described in WO2015058206; WO2014004577; WO2006108101; and WO2008130977. The methods can include enrichment of the sample for organ-derived cells by depletion of other cells, e.g., removal of red blood cells and platelets, depletion of peripheral white blood cells, and recovery of liver cells.

Depletion of contaminating white blood cells can include the application of biotin labeled depletion antibodies to blood which are subsequently incubated with streptavidin magnetic beads prior to microfluidic processing. These depletion antibodies select cell types which will be removed from the product during magnetic sorting. An exemplary white blood cell depletion cocktail includes antibodies to CD45, CD16, and CD66b. Commercially available biotin labeled antibodies against a range of lineage-committed antigens can be used, e.g., including antibodies directed against CD2, CD3, CD11b, CD14, CD15, CD16, CD19, CD56, CD123, CD235a (Miltenyi Biotec). Further, since these antibodies are also biotin labeled, they can simply be substituted to the magnetic bead incubation step prior to microfluidic sorting.

The cells that can be evaluated in the present methods can include organ specific immune cells and structural cells.

Immune cells may migrate from or perhaps infiltrate in the organ during rejection, which may respectively cause higher or lower levels of these cells in the peripheral blood. Examples include T lymphocytes and dendritic cells.

If there is injury to the organ caused by the rejection then higher levels of organ specific structural cells may be present in the peripheral blood. For example endothelial cell for all organs. Or specific cells like cadiomyocytes for hearts, hepatocytes for liver, tubulus cells for kidneys etc.

The present methods can also include detecting specific nucleic acids or proteins from donor-derived cells, e.g., using a multiplex panel for detecting one or more transcripts, e.g., as listed in Table A for livers. Other biomolecules can readily be identified that indicate identity, status, or activity of a selected cell type.

TABLE A
Cells of interest and corresponding specific transcript
for amplification using ddPCR multiplex assay.

| Cell of | Specific | |
|---|---|---|
| origin | Transcript | Clinical relevance |
| Endothelial | Clec3m (AKA | Degree of vascular endothelial damage (or |
| cells | L-Sign) | “endothelitis”) is used to diagnose ACR. |
| Stellate cells | α-SMA, Lrat, | Stellate cell activation is correlated to |
| | Desmin | acute rejection episodes. |
| Kupffer cells | Clec4f | Antigen-presenting cells which play a role |
| | | in graft rejection; important role in |
| | | ischemia-reperfusion injury which is the |
| | | major cause of primary non-function and |
| | | early allograft dysfunction. |
| Hepatocytes | Albumin (Alb), | Hepatic steatosis is one of the most |
| | Fatty acid | important variables in determining graft |
| | binding protein | function after transplantation. |
| | 1 (fabp1) | |

Additionally, the transcripts can include Stab1, Lyve1, Vap-1 for hepatic sinusoidal endothelial cells⁵⁶, Lrat and Desmin for stellate cells²³, and alpha 2-HS glycoprotein, apolipoprotein H, fibrinogen beta chain⁴⁷ for hepatocytes.

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of the transcripts. Measurement of the level of a biomarker can be direct or indirect. For example, the abundance levels of the transcripts can be directly quantitated.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, e.g., glyceraldehyde-3-phosphate dehydrogenase (GAPDH), beta-actin (ACTB), TATA-binding protein (TBP), ribosomal proteins (RP), are commonly used (Waxman and Wurmbach, BMC Genomics. 2007; 8: 243).

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, droplet digital PCR (ddPCR) is used.

In some embodiments, the presence and/or level of a graft-derived cell or transcript marker is comparable to the presence and/or level of the marker(s) in the disease reference, and the subject has one or more symptoms associated with rejection or injury, then the subject is rejecting the graft or has an injury. In some embodiments, the subject has no overt signs or symptoms of rejection or injury, but the presence and/or level of one or more of the proteins evaluated is comparable to the presence and/or level of the protein(s) in the disease reference, then the subject has an increased risk of rejection or injury. In some embodiments, once it has been determined that a subject is rejecting a graft or has an increased risk of developing rejection or injury, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of a marker, e.g., a control reference level that represents a normal level of the marker, e.g., a level in a subject (or cohort of subjects) who is not rejecting their graft and not at risk of developing graft rejection or injury, and/or a disease reference that represents a level of the proteins associated with conditions associated with graft rejection or increased graft rejection or injury, e.g., a level in a subject (or cohort of subjects) who are undergoing or later undergo graft rejection (e.g., with 180, 90, 60, 30, 14, or 7 days).

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects.

A disease reference subject is one who has (or has an increased risk of developing) acute or chronic graft rejection or injury. An increased risk is defined as a risk above the risk of subjects in a population of graft recipients.

In characterizing likelihood, or risk, numerous predetermined values can be established.

If the subject is determined to be undergoing rejection or at risk for acute or chronic rejection or injury, the methods can include administering a treatment to the subject for rejection or to reduce the risk of rejection or injury. Treatments can include administration of immunosuppressant drugs, e.g., from one or more classes of maintenance drugs, including calcineurin inhibitors (e.g., tacrolimus and cyclosporine); antiproliferative agents (e.g., mycophenolate mofetil, mycophenolate sodium, and azathioprine); mTOR inhibitors (e.g., Sirolimus); and steroids (e.g., prednisone). See, e.g., Allison et al., Nurs Clin North Am. 2016 March; 51(1):107-20; Stolp et al., Methods Mol Biol. 2019; 1899:159-180.

Monitoring Viability of Livers Pre- and Post-Transplant

End-stage liver disease contributes to 77,000 deaths annually in the US alone¹ and transplantation is often the only treatment option. Due to the severe donor organ shortage, merely 12,000 patients are listed on the liver transplant wait-list and of these, only 8,000 will receive a transplant each year in the US². Thus, improving access to this lifesaving treatment has become an immediate necessity. Paradoxically, the donor organ shortage is not caused by a limited availability of cadaveric organs more than 25% of donor livers procured for transplantation are not ultimately transplanted³. Also, it is estimated that there is an additional donor pool of 6,000 unprocured livers/year, of which many are only marginally damaged⁴. However, because of their uncertain viability, none of these potential donor organs are used while transplantation of just a fraction of these organs could dramatically reduce the organ shortage.

Liver-specific cell types can be categorized as structural or resident immune cells, and both could be promising candidates for assessing organ injury. Structural liver cells such as liver sinusoidal endothelial cells (LSECs), hepatocytes, and liver stellate cells typically stay in the liver under normal physiological conditions. However, upon liver injury, it was hypothesize that they would be likely to be released due to their anatomical location near the sinusoidal capillaries¹². Further, the liver is home to three types of resident immune cells^{13,14}: 1) Kupffer cells, 2) liver-specific natural killer cells (also known as pit cells¹⁵), and (3) dendritic cells. Because tissue injury is either primarily caused by or secondarily evokes an immune reaction¹⁶⁻¹⁸, detectable alterations in the immune cells that are released from the organ may correlate with tissue injury and organ viability. Thus, for liver injury the present methods can also include detecting the presence of hepatocytes (HC), liver sinusoidal endothelial cells (LSEC), Kupffer cells (KC), hepatic stellate cells (SC), and/or dendritic cells (DC), while also differentiating against peripheral cells, e.g., using one or more cell surface markers listed in Table B.

TABLE B
Markers for identifying liver-derived cells using imaging flow cytometry.

| | GLUT 2 | CD32 | CD34 | CD36 | CD45 | CD68 | CD146 | CD200 |
|---|---|---|---|---|---|---|---|---|
| HC | + | − | − | + | − | − | − | − |
| SEC (type 1) | − | − | − | + | − | − | − | − |
| SEC (type 2) | − | + | − | − | − | − | − | − |
| KC | − | + | − | + | + | + | − | − |
| HSC | − | − | − | + | − | − | + | − |
| HSC (act) | − | − | − | + | − | + | + | − |
| DC | − | − | − | + | − | − | − | + |
| WBC | − | + | − | + | + | + | + | + |
| VEC | − | + | + | + | − | − | + | + |
| Platelets | − | + | − | + | − | − | − | − |
| Hemo stemcell | − | − | + | + | − | − | − | − |

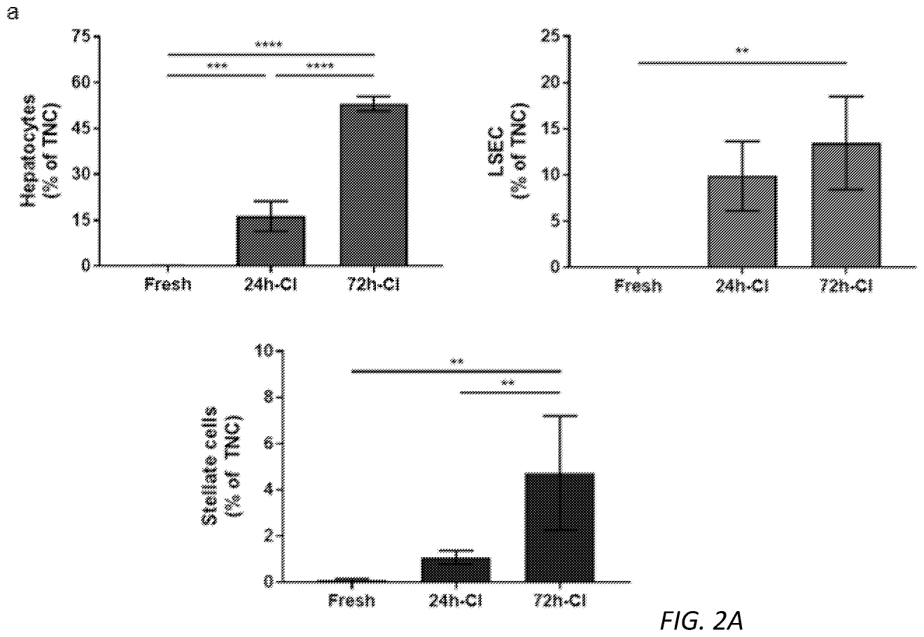

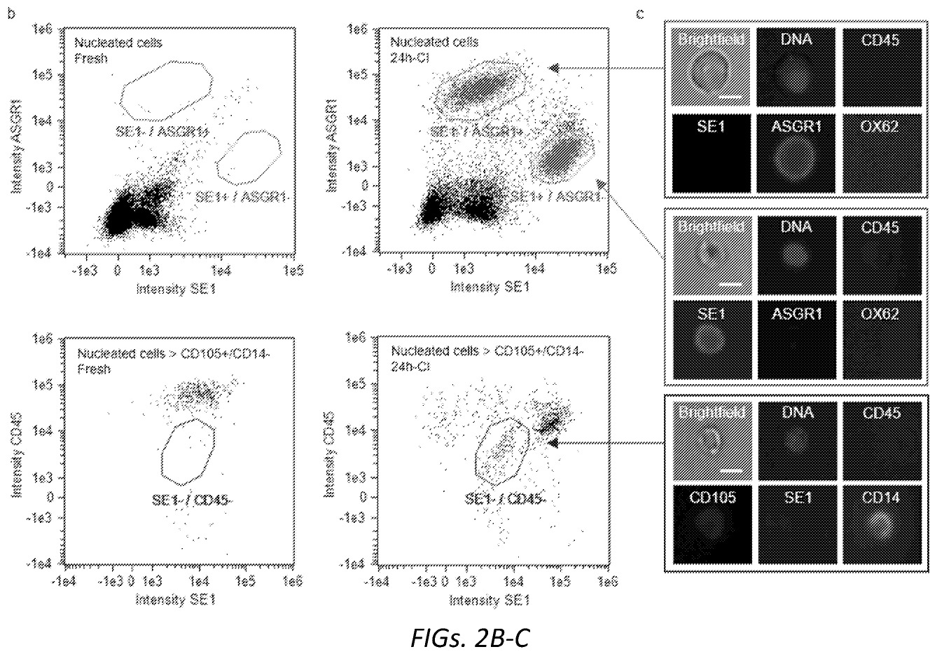

The present results confirmed that tissue injury during preservation leads to the release of structural liver cells (FIGS. 2A-C) and alterations in the release of liver-resident immune cells (FIGS. 3A-C). Although endothelial cell detachment has been studied as a marker of vascular injury⁴⁷, we show here for the first time, that parenchymal cells are also released from organs under non-proliferative pathological conditions.

The cell release data presented herein is reported as a percentage of the TNCs instead of absolute numbers of specific cell types (although these data are also relevant and have been provided in Table 3). Such an approach has multiple important advantages. First, it reduces the standard error of liver-to-liver variances in overall cell release. Second, the percentage of TNCs provides a metric that is independent of liver size and perfusate volume. Such metrics facilitate the translation from animal models to human studies because variation in organ size does not change the percentage of liver specific cells whereas it most likely will change the absolute numbers of released cells. The size indifference is additionally important for translational efforts because human livers vary up to 300% in liver weight between donors⁴⁸, and different flush and perfusate volumes are used in clinical protocols.

Although the released cells during perfusion also included remnant peripheral white blood cells, a substantial proportion (˜20-75% dependent on liver viability; FIGS. 2A and 3A) of the released cells were liver specific and included both structural and resident immune cells. The identity of these liver specific cell population was presumed based on surface marker expression as indicated by superscript p throughout this disclosure. For the structural cells (FIGS. 1A-C), we focused on hepatocytes, LSECs, and stellate cells and were guided by our hypothesis that they were most likely to be released through their pericapillary anatomical localization. The three cell types were nearly absent in the perfusate of fresh livers and all three significantly increased as a function of cold ischemic injury. Most importantly, in our ex vivo model, the release of hepatocytes and stellate cells during perfusion discriminated fresh, mild and severe ischemic livers with strong statistical significance (FIG. 1B).

Of all the liver-specific structural cell releases that were measured during perfusion, hepatocyte release was the most sensitive marker of ischemic injury during hypothermic preservation. This may be due to the large number of hepatocytes in livers. Based on previous literature, rat livers contain about 1.4×10⁹ hepatocytes⁵⁰. This is over one thousand times the number of hepatocytes that were released after severe ischemia, and over one million times the number of hepatocytes that were released from fresh livers (Table 3). This indicates that hepatocyte release can be detected with high sensitivity. As a corollary question, we analyzed if hepatocyte release was related to the alterations in hepatocyte function and injury as result of cold ischemia. We found highly significant correlations between hepatocyte release and bile production, AST release, and oxygen uptake during perfusion. This demonstrates that specific markers of hepatocyte and function may be reflected by hepatocyte release.

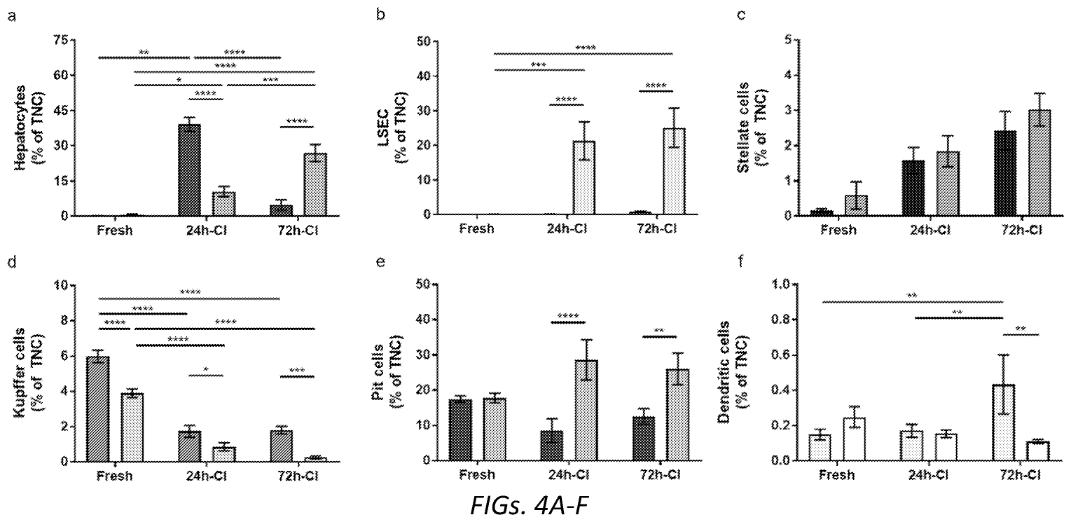

We found that the percentages of liver-resident immune cells that were released in the perfusate showed a declining trend with increasing durations of ischemia (FIGS. 3A-C), as opposed to the above discussed increasing numbers of structural cells (FIGS. 2A-C). This effect was strongest for the Kupffer cell percentages which were highest in the perfusates of fresh livers and significantly decreased as a function of increasing CI time. This may be explained by Kupffer cell activation and infiltration during cold ischemia. Kupffer cells have an important mediating role in ischemia-reperfusion (I/R) injury, which is a major cause of primary non-function and early allograft dysfunction⁸. While machine perfusion has shown to reduce IR injury during transplantation via multiple pathways, one of the proposed mechanisms is that cytokines are diluted in the perfusate. This results in reduced Kupffer cell activation and infiltration⁵⁹, as was seen in histology samples of hypothermic preserved and machine perfused grafts⁶⁰. However, in the present study, we showed that Kupffer^{p} cells were washed-out during machine perfusion (FIG. 4D and Table 3), which may be another reason for reduced I/R injury after machine perfusion.

Thus, the present methods can include detecting the presence or number (e.g., as a percentage of cells) of liver specific cells, and identifying an organ as non-viable or a subject as undergoing or at risk of undergoing rejection, when the presence or number of liver specific cells is above a reference level.

Monitoring Viability of Kidneys Pre- and Post-Transplant

To determine the immunosuppressive state of a renal graft, we sample peripheral blood to isolate and characterize cells derived from the graft. Culminating evidence supports circulation of donor whole cells, as well as microparticles or the remnants of lysed cells which release cellular organelles into the recipient circulation (Rutkowska et al. Ann. Transplant. 12, 12-14 (2007)). In the case of sex mismatched male to female transplantation, researchers used primers targeted towards the Y-chromosome to detect donor DNA in recipient blood after gut (Morin et al., Transplant. Proc. 32, 1290-1291 (2000); Okuda et al., Transplant. Proc. 32, 1278 (2000)), kidney (Inman et al. Transplantation 67, 1381-1383 (1999); Wang et al. Transplant. Proc. 33, 177-178 (2001).), and liver (Tashiro et al., Hepatol. Baltim. Md. 23, 828-834 (1996)) transplantation. Further, many have shown vascularized organs shed passenger cells from the transplanted organ which migrate through the recipients circulatory system (Rutkowska et al. Ann. Transplant. 12, 12-14 (2007); Rao et al., Curr. Opin. Nephrol. Hypertens. 3, 589-595 (1994)). Furthermore, it is general believed that migratory cells leave the graft and present donor antigens to the host, which can lead to allograft rejection (Larsen et al., Ann. Surg. 212, 308-317 (1990)). Finally, evidence suggests donor-derived biologics may remain in the recipient's circulation for substantial periods of time.

To assay for renal injury or impending rejection, the methods can include detecting renal-specific cells including endothelial cells, mesangial cells, podocytes, renal pariental cells, juxtaglomerulal cells, proximal tubule cells, thick ascending limb cells, and distal tubule cells, while also differentiating against peripheral white blood cells (WBC), other vascular endothelial cells (VEC), platelets, and hemopoietic stem cells, e.g., using one or more cell surface markers listed in Table C.

TABLE C
Markers for identifying kidney-derived
cells using imaging flow cytometry.

| Location | Cells | Marker |
|---|---|---|
| Glomerulus | Mesangial Cells | PDGFβ-R |
| | Podocytes | Nephrin |
| | | Podocin |
| | | NEPH1 |
| Bowman's | Renal Pariental Cells | WT-1 |
| capsule | Juxtaglomerulal Cells | renin |
| Tubules & Henles | Proximal Tubule Cell | SLC22A8 |
| loc | | SLC22A13 |
| | Thik Ascending Limb Cells | ROMK |
| | Distal Tubule Cell | Uromodulin (UMOD) |
| | | SLC12A1 |
| | | SLC12A1 |

### EXAMPLES

The invention is further described in the following examples, which do not limit the scope of the invention described in the claims.

Materials and Methods

The following materials and methods were used in the Examples below.

Experimental Design

The donor livers were procured and subjected to HP for 0 (fresh) (n=4), 24 h (n=5), and 72 h (n=4), after which the livers underwent 3 h of SNMP. Allocation of the livers was arbitrarily alternated between the experimental groups. The perfusate was collected and analyzed using multi-channel imaging flow cytometry to detect hepatocytes, sinusoidal endothelial, stellate, Kupffer, pit (liver-associated natural killer), and dendritic cells The first 100 ml and last 100 ml of perfusate that flushed through the livers were collected separately from the 400 ml recirculating perfusate to study the changes in cell-release profiles (FIGS. 1A-C).

Animal Care

Livers from healthy male Lewis rats (Charles River Laboratories, Wilmington, Mass., USA) were used for all experiments to ensure comparable baseline characteristics between the experimental groups (Table 1). The animals were housed in a temperature- and humidity-controlled room equipped with a natural light/dark cycle, socially housed (according to weight standards) with conventional bedding, and were provided with free access to standard food and water—in accordance with the National Research Council guidelines. The health and welfare of the animals was maintained by Massachusetts General Hospital Center of Comparative Medicine (CCM), and the experimental protocols were approved by the Institutional Animal Care and Use Committee (IACUC) of Massachusetts General Hospital (Boston, Mass., USA).

TABLE 1
Animal characteristics of the experimental groups

| | Fresh | 24 h-CI | 72 h-CI |
|---|---|---|---|
| Characteristic | (mean ± SD) | (mean ± SD) | (mean ± SD) |
| Group size (n) | 4 | 5 | 4 |
| Species, Strain | Rattus, Lewis | Rattus, Lewis | Rattus, Lewis |
| Sex (% male) | 100% | 100% | 100% |
| Age (weeks) | 14.4 ± 1.2 | 13.9 ± 1.2 | 14.3 ± 1.1 |
| Liver weight (grams) | 12.4 ± 1.6 | 13.4 ± 1.1 | 12.9 ± 1.7 |

Liver Procurement

The rats (200-250 g) were anesthetized with isoflurane in a dedicated animal surgery laboratory room. The liver was exposed through a transverse abdominal incision and freed from its connecting ligaments. The bile duct (BD) was dissected and cannulated with a 28-gauge catheter using a surgical microscope. Next, the portal vein (PV) was cannulated past the gastroduodenal and splenic branches with a 16-gauge catheter. The cannulas of the PV and BD were secured using 3.0 silk sutures. After the hepatic artery was identified and tied off, the infrahepatic inferior vena cava (IHVC) was transected and the liver was flushed with 60 ml of ice-cold saline. While still under anesthesia, the rats were euthanized by exsanguination from the IHVC. Finally, the suprahepatic inferior vena cava and hepatoduodenal ligament were transected, and the liver was freed from the remaining ligaments and removed from the abdomen.

Hypothermic Preservation

Directly after procurement, the liver for the 24-h-CI and 72-h-CI groups were flushed with 30 ml of ice-cold University of Wisconsin (UW) solution (Bridge to Life, Columbia, S.C., USA). Next, the livers were submerged in UW solution and stored at 4° C. in a sealed bag.

Subnormothermic Machine Perfusion

The machine perfusion system consists of non-pulsatile circulation providing portal perfusion through the liver. The perfusate is pumped from a 500-ml reservoir bottle by a flow-rate controlled roller pump (Cole Palmer, Vernon Hills, Ill., USA) through an oxygenator (Radnoti, Monrovia, Calif., USA), bubble trap (Radnoti), pressure sensor (Living Systems Instrumentation, Albans City, Vt., USA), sampling port (Cole Palmer), and finally, an organ chamber that holds the liver during perfusion. The components are connected in consecutive order with size-16 silicone tubing (Cole Palmer). The oxygenator is supplied with a gas mixture of 95% 02 and 5% CO₂, and the perfusion temperature is maintained at an ambient temperature of 21° C. (±1° C.).

Prior to perfusion, the system was primed with 500 ml of perfusate. The perfusate consisted of powdered Williams Medium E (Sigma-Aldrich, St Louis, Mo., USA) supplemented with sodium bicarbonate (2.2 g/l; Sigma-Aldrich), dexamethasone (24 mg/l; Sigma-Aldrich), insulin (5 U/l; MGH Pharmacy), heparin (2000 U/l; MGH Pharmacy), and bovine serum albumin (10 mg/ml; Sigma-Aldrich). The bubble trap was filled to 25% with perfusate and therefore also served as a compliance chamber to minimize pressure pulses created by the roller pumps. The system was run freely for ˜15 min to oxygenate the perfusate and adjust the pH to 7.3-7.4 with sodium bicarbonate, if necessary. During this period, pressure sensor was calibrated using a dummy 16-gage catheter (the same cannula that was used to cannulate the PV) for flow rates from 0 to 30 ml/min.

The perfusion was initiated by attaching the PV cannula to the perfusion system. The outflow of the BD cannula was placed in a 1.5-ml Eppendorf tube to collect the produced bile. During the first 30 min of perfusion, the pressure over the liver was gradually increased to 5 mmHg and regulated throughout perfusion by manually adjusting the flow rate of the pump, up to a flowrate of maximum 25 ml/min. The liver drained freely into the organ chamber and the first 100-ml return of the organ chamber was collected in a 100-ml graded cylinder before closing the circuit. The remaining perfusate was recirculated by directing the return of the organ chamber to the perfusate reservoir that closed the perfusion circuit. After 3 h of perfusion, the perfusate reservoir was replaced with a fresh perfusate bottle. This created a small bubble in the system that was followed until it was caught in the bubble trap. At this moment, the return of the organ chamber was diverted into a fresh bottle, and the last 100 ml of fresh perfusate was collected separately.

Imaging Flow Cytometry

The hepatocytes, LSECs, stellate, Kupffer, pit, and dendritic cells in the perfusates were studied using the ImageStreamX Mark II imaging flow cytometer (Amnis Corporation, Seattle, Wash., USA) equipped with 405-, 488-, and 642-nm lasers, a 40× objective, and six imaging channels. To identify the six different cell types, we used three flow cytometry panels for the detection of two cell types each: panel 1 for the detection of hepatocytes and LSECs; panel 2 for the detection of stellate cells and Kupffer cells; panel 3 for the detection of pit cells and dendritic cells.

Within 1 h after the end of SNMP, half the volumes of the three perfusates were spun down at 200 g and resuspended in a total volume of 1.5 ml. For each of the three perfusate fractions, three 200-μl aliquots of the 1.5-ml cell suspension were stained for the different flow cytometry panels as follows:

All aliquots were stained with DRAQ (1:200; Biolegend, Deadham, Mass., USA; Cat #424101) and PECy7-conjugated CD45 antibody (1:50; Biolegend, Cat #202214). The aliquot for panel 1 was additionally stained for the detection of hepatocytes^{p} and LSECs^{p} with Alexa 405-conjugated SE1 (1:100; Novus Biologicals, Centennial, Colo., USA; Cat #NB110-68095AF405), FITC-conjugated ASGR1 (1:50; Miltenyi Biotec, Cambridge, Mass., USA; Cat #130-109-490), and PE-conjugated OX62 (1:100; Thermo Fisher, Waltham, Mass., USA; Cat #12-1030-82) antibodies.

The aliquot for panel 2 was additionally stained for the detection of stellate^{p} and Kupffer^{p} cells with Alexa 405-conjugated CD105 (1:87; Novus Biologicals; Cat #NB500-452AF405), FITC-conjugated SE1 (1:100; Novus Biologicals; Cat #NB110-68095F), and Cy3-conjugated CD14 (2:100; Bioss, Woburn, Mass., USA; Cay #bs-1192R-Cy3) antibodies.

The aliquot for panel 3 was additionally stained for the detection of pit cells and dendriticp cells with Alexa 405-conjugated NKRP1A (1:80; Novus Biologicals; Cat #NB100-65297AF405), FITC-conjugated CD3 (1:50; Invitrogen, Carlsbad, Calif., USA; Cat #11-0030-82), and PE-conjugated OX62 (1:100; Thermo Fisher; Cat #12-1030-82) antibodies.

All stains were incubated at room temperature for 30 min except DRAQ, which was incubated for 10 min. Directly after incubation, each of the total of nine samples per perfusion was run separately in the imaging flow cytometer with the laser powers set at 100, 100, and 150 mW for the 405-, 488-, and 642-nm lasers, respectively.

Imaging Flow Cytometry Data Processing

Flow cytometry data was processed using the IDEAS 6.2 (Amnis Corporation) software package. Out of all the recorded events, we selected the normal-shaped and -sized events (NSS) using area and aspect ratio of the brightfield channel. Hence only whole cells are included in subsequent analysis and smaller events such as cell debris or extracellular vesicles are excluded. Out of these NSS events, we selected the DRAQ-positive events to obtain the TNCs. To select the cells of interest more accurately, we first selected all TNCs that were positive for at least one of the remaining channels and used this potential cell-of-interest (PCI) population for further analysis.

For panel 1, we selected the hepatocytes^{p} as ASGR1⁺/SE1⁻ cells from the PCI population and confirmed that those cells were CD45− and OX62−. LSECs were selected from the same PCI population as ASGR1⁻/SE1⁺ cells and confirmed that they were also CD45− and OX62−.

In panel 2, we selected the stellate^{p} cells in two steps: first, we gated CD105⁺/CD14⁺ cells from the PCI population; then, we selected the stellate cells as CD45⁻/SE1⁻ cells from this subpopulation. To select the Kupffer^{p} cells, we gated the CD14⁺/CD105⁻ population and confirmed that these cells were CD45⁺ and SE1⁻. Because CD14 expression is influenced by Kupffer cell activation^{30,32} we used an intensity threshold of 1e4 to define CD14⁺ cells, as illustrated in FIG. 2b, top.

In panel 3, we selected the pit^{p} cells as the NKRP1A⁺/CD3⁻ cells from the PCI population and confirmed that those cells were CD45⁺ and OX62⁻. We selected the dendriticp cells from the PCI in three steps. In the first two steps we selected the NKRP1A⁻/CD3⁻ cells from the CD45⁺/OX62⁺ cells in the PCI population. Because dendriticp cells are rare, we then manually selected the true CD45⁺/OX62⁺/NKRP1A⁻/CD3⁻ cells from this subpopulation.

Relative numbers of the specific cell types in the perfusate were calculated by dividing the number of each cell type by the number of TNCs in each panel. Absolute numbers of the specific cell types in the perfusate were calculated as follows: the count of each cell type was divided by the volume processed by the flow cytometer to obtain the specific cell concentrations in the 200-μl flow cytometry aliquot. This concentration was multiplied by the centrifugation dilution factor and by the total volume of the perfusate (i.e., either 100 or 400 ml) to obtain the total number of specific cell types in the perfusate.

Optimization and Validation of Imaging Flow Cytometry Analysis

For optimization of the imaging flow cytometry analysis, we verified specificity of the antibodies and optimized antibody concentration as well as incubation time using purified populations of rat liver cells obtained from a two-step EDTA collagenase procedure from the Cell Resource Core (Massachusetts General Hospital, Boston, Mass., USA) as per established protocols^{65,66}. In this capacity, positive and negative controls were derived from purified cell fractions of either hepatocytes, LSECs, stellate cells, Kupffer cells, or a fraction containing all non-parenchymal cells for identification of Pit and dendritic cells. Further, we also assessed the influence of other variables on antibody performance including different antibody clones, fluorophores, and vendors.

Perfusion Data Acquisition

The liver was weighed directly after procurement, HP, and SNMP. Real-time perfusate measurements were performed every 30 min; pH and pO₂ were measured in the PV and IHVC, and the potassium concentration was only measured in the IHCV using an i-STAT blood analyzer (Abbot Laboratories, Chicago, Ill., USA). Additional perfusate samples were taken from the IHVC, immediately frozen on dry ice, and stored at −80° C. for post-hoc analysis of AST activity using a colorimetric kit (ThermoFisher Scientific, Pittsburgh, Pa.) according to the manufacturers' instructions. Cumulative bile production was measured by weighing the bile-containing Eppendorf tube on a microscale.

For histological analysis, tissue biopsies were taken directly after perfusion, fixed in buffered 5% (v/v) formaldehyde for 24 hours, and stored in 70% (v/v) ethanol. Tissue processing and staining for hematoxylin and eosin (H&E) was performed at the Massachusetts General Hospital Histology Molecular Pathology Core Facility, Boston, Mass., USA. Histology slides were assessed by an experienced liver pathologist (E.O.A.H). For analysis of adenylate energy charge, additional (approx. 1 gr) tissue biopsies were flash frozen in liquid nitrogen and stored at −80° C. Measurement of the adenylate energy charge by liquid chromatography-mass spectrometry (LC-MS) was performed at the Shriners Hospitals—Boston Mass. Spectrometry Core Facility, Boston, Mass.

Perfusion Data Processing

To calculate vascular resistance in the PV, the perfusion pressure was divided by the corresponding flow rate that was multiplied by the weight of the liver after procurement.

Oxygen concentrations in the outflow (IHVC) and the inflow (PV) were derived from Henry's law, CdO₂=aO₂×PO₂, where CdO₂ is the concentration of dissolved oxygen, aO₂ is the solubility coefficient for oxygen (0.00314 ml 02/mmHg O₂/dl blood), and PO₂ is the partial oxygen pressure that was measured in the PV and IHVC during SNMP. To calculate the oxygen uptake rate (OUR), oxygen concentration of the outflow was subtracted from that of the inflow; this difference was multiplied by the flow rate and finally divided by the weight of the liver after procurement.

Statistical Analysis

Repeated measures two-way ANOVAs were used for the comparison of the time-course perfusion and cell-release data, followed by Tukey's post-hoc test to examine statistical differences between the experimental groups and to correct for multiple comparisons. Total oxygen uptake was calculated by integration of the oxygen uptake rate to calculate the area under the curve (AUC). Total oxygen uptake was compared with the Kruskal-Wallis test, followed by Dunn's post-hoc test. Linear regressions were used to correlate the viability parameters to cell release during machine perfusion. All statistical analyses were performed with Prism 7.03 (GraphPad Software Inc., La Jolla, Calif.) with a (two-sided) significance level of 0.05. Adequacy of the statistical analysis was endorsed by an experienced Biostatistician (A.M.).

Human liver cell isolation. Initial testing and optimization is done with human liver cells isolated from discarded whole human liver grafts and subsequently spiked into whole blood to optimize the technology. Protocols for isolation of human liver cells include protocols described in^{23,24} via a standard two-step collagenase protocol²⁵. Cells obtained from each liver are split into five fractions: 1) purified hepatocytes, 2) non-parenchymal cells (NPC), 3) stellate cells (SC), 4) liver sinusoidal endothelial cells (LSEC), and 5) Kupffer cells (KC). Protocols are detailed elsewhere²⁴ and have already been tested by the Cell Resource Core.

Blood collection & microfluidic isolation of liver-derived cells. Fresh healthy donor blood will be collected in ACD-A anticoagulant tube. For spiked cell experiments (1-1,000 cells/mL blood), human liver-derived cells (isolated as described above) are added directly to the blood collection tube to serve as a model. For enumeration experiments of spiked cells, CellTracker (Life Technologies)-labeled human liver cells are spiked into healthy donor blood (note: transplant patient blood specimens are used without spiking). Spiked blood samples are incubated with biotinylated antibodies for magnetophoresis, followed by a second incubation with streptavidin-coupled Dynabeads (Invitrogen), and loaded into a pressurized syringe for microfluidic processing, as described below. First, nucleated cells are separated from other blood components, e.g., using microfluidic rheology-based cell sorting and leukocytes are labeled with antibody-conjugated magnetic beads and collected with a magnet (“magnetophoresis”). Spiked cells are enumerated by counting CellTracker-positive cells in two Nageotte chambers (Hausser Scientific). Leukocytes will be enumerated as nucleated cells that were not spiked cells (positive for DyeCycle green, negative for Cell Tracker red).

### Example 1. Total Cell Release During Machine Perfusion as a Function of Cold Ischemia Duration

The clinical standard for organ preservation is hypothermic preservation (HP) at 4° C. in a specialized preservation solution such as the University of Wisconsin solution (UW)¹⁹. For rat livers, the maximum viable HP duration is 24 h²⁰. We have previously shown that extending the duration of CI leads to a sharp decline in organ viability, resulting in 0% transplant survival after 72 h of HP, despite a 3-h subnormothermic machine perfusion (SNMP) resuscitation^{20,21}. Therefore, we chose to study cell release from rat lives after these two CI durations to represent transplantable (24-h-CI) vs. non-transplantable (72-h-CI) rat livers, in addition to a fresh control. Following CI, all livers were subjected to 3-h SNMP. We isolated cells from the perfusate that recirculated during SNMP and analyzed them using imaging flow cytometry. We refer to this as cell release “duringperfusion” and the corresponding data is shown in FIGS. 1B, 2A-C, and 3A-C. Additionally we flushed the livers with separate fresh perfusate fractions at the “start of perfusion” and “end of perfusion”, to study the cell release in the flush directly after HP and the difference in cell release over time. The corresponding data is shown in FIGS. 1C and 4A-F. The research design is schematically shown in FIG. 1 and explained in detail in the Materials and Methods.

We first studied if and how many cells were released from fresh and injured liver grafts by analyzing the total number of cells released from fresh, 24-h-CI and 72-h-CI livers during perfusion. We found that over 3 million cells were released from fresh (n=4) and 24-h-CI (n=5) livers during perfusion (3.64±0.74 million (M) and 3.40±1.63 M, respectively; mean±standard deviation (SD) throughout the text, unless specified), providing ample cell quantities for downstream analysis (FIG. 1B). Cell release from 72-h-CI (n=4) livers was significantly lower (1.94±0.95 M; repeated measures two-way ANOVA followed by the Tukey post-hoc test throughout the text unless otherwise specified) than fresh and 24-h-CI livers (p=0.0114 and p=0.0225, respectively). Further, we found that white blood cells (i.e. CD45+ cells) accounted for a considerable percentage of cells that declined in number as a function of CI time (94.6±5.92%, 51.2±19.65%, 15.8±6.27% in fresh, 24-h-CI, 48-h-CI, and 72-h-CI livers, respectively; Table 2). We suggest that a significant portion of this white blood cell population originates from peripheral blood rather than from the liver because only ˜13-21% of the released cells were identified as liver-specific immune cells.

TABLE 2
Relative surface marker expression on all released nucleated cells

| | Fresh | 24 h-CI | 72 h-CI |
|---|---|---|---|
| Surface Marker | % (Mean ± SD) | % (Mean ± SD) | % (Mean ± SD) |
| CD45 | 94.59 ± 5.92 | 51.20 ± 19.65 | 15.77 ± 6.27 |
| ASGR1 | 0.48 ± 0.48 | 19.22 ± 11.98 | 57.45 ± 5.29 |
| SE1 | 0.23 ± 0.23 | 11.16 ± 9.34 | 15.43 ± 10.60 |
| CD105 | 4.36 ± 2.20 | 18.29 ± 8.73 | 45.10 ± 21.72 |
| CD14 | 8.98 ± 2.31 | 22.48 ± 11.43 | 49.94 ± 22.48 |
| NKRP1A | 18.23 ± 2.33 | 17.99 ± 10.22 | 22.55 ± 8.14 |
| CD3 | 45.75 ± 7.54 | 25.10 ± 10.39 | 13.57 ± 9.44 |
| OX62 | 2.38 ± 0.29 | 4.21 ± 1.67 | 9.16 ± 4.98 |

Note:
Percentage is relative to the total number of nucleated cells that are released into the perfusate of fresh, 24-h-cold ischemic (CI), and 72-h-CI livers. Cells can express more than one marker.

### Example 2. Release of Structural Liver Cells During Machine Perfusion

After confirming that millions of cells are released during perfusion, we used imaging flow cytometry to characterize the cell types released from the liver grafts during perfusion. Cell type numbers were expressed and analyzed as a percentage of the total number of nucleated cells (TNCs) released in the perfusate (the absolute values are presented in Table 3). For analyzing structural liver cells, we used ASGR1 as a marker for hepatocytes²²⁻²⁴; however, it should be noted that 5-10% of the ASGR1-positive cells have been reported as fibroblasts in human liver biopsies²⁴. For LSECs, we used the rat-specific sinusoidal endothelial cell marker SE1^{25,26}. Stellate cells express CD105²⁷; however, this surface marker can also be expressed on LSECs^{28,29}. Therefore, CD105-positive and SE1-negative cells were selected as stellate cells (FIG. 2C). ASGR1+, SE1+, and CD105+/SE1− cells are referred to as presumed (denoted by p in superscript) hepatocytes, LSECs, and stellate cells, respectively in the text.

All three types of structural liver cells were nearly absent in the perfusates of fresh livers. After 24 h and 72 h of CI, increasing numbers of all three types of structural liver cells were released into the perfusate (FIG. 2A). Intensity plots of the specific surface markers (FIG. 2B) clearly showed distinct hepatocyte, LSEC, and stellate cell populations in the CI groups, but not in the fresh controls.

This increased release of structural liver cells after CI was most evident for hepatocytes, which showed significantly different percentages in the perfusates of fresh (0.12±0.05%), 24-h-CI (16.32±11.01%) and 72-h-CI (53.06±4.70%) livers (p<0.0001 between all groups). LSEC release showed a similar trend, with a significantly higher percentage of LSECs in the perfusates of 72-h-CI livers (13.46±10.05%) compared with that of fresh (0.01±0.00%) livers (p=0.0350). The percentages of released stellate^{p} cells (FIG. 2A) were much lower (0.11±0.11%, 1.08±0.64% and 4.74±4.94% for fresh, 24-h-CI, and 72-h-CI livers, respectively) than those of hepatocytes and LSECs. However, a significant increase in the percentage of stellate cells was found in 72-h-CI livers compared with fresh and 24-h-CI livers (p=0.0019 and p=0.0099, respectively). Together, these results confirm that tissue injury leads to the release of structural liver cells. More importantly, it shows that the release of structural liver cells is significantly different between livers with no, mild, and severe ischemic injury.

TABLE 3
Absolute number of liver-specific cells released into the perfusate

| | Fresh | 24 h-CI | 72 h-CI |
|---|---|---|---|
| Cell type | (Mean ± SD) | (Mean ± SD) | (Mean ± SD) |
| Hepatocytes | 4.47 × 10³ ± 1.95 × 10³ | 6.49 × 10⁵ ± 6.76 × 10⁵ | 1.18 × 10⁶ ± 5.44 × 10⁵ |
| LSECs | 3.74 × 10² ± 2.72 × 10² | 2.66 × 10⁵ ± 2.09 × 10⁵ | 2.56 × 10⁵ ± 2.65 × 10⁵ |
| Stellate Cells | 4.49 × 10³ ± 5.43 × 10³ | 3.37 × 10⁴ ± 2.72 × 10⁴ | 5.55 × 10⁴ ± 3.54 × 10⁴ |
| Kupffer Cells | 1.55 × 10⁵ ± 4.47 × 10⁴ | 2.05 × 10⁴ ± 8.64 × 10³ | 6.49 × 10³ ± 4.67 × 10³ |
| Pit Cells | 5.72 × 10⁵ ± 1.14 × 10⁵ | 4.95 × 10⁵ ± 2.01 × 10⁵ | 3.21 × 10⁵ ± 2.68 × 10⁵ |
| Dendritic Cells | 4.67 × 10³ ± 1.58 × 10³ | 2.32 × 10³ ± 3.58 × 10² | 1.67 × 10³ ± 8.22 × 10² |

Note:
CI = cold ischemia.

### Example 3. Release of Liver-Resident Immune Cells During Machine Perfusion

To analyze the percentages of liver-resident immune cells in the perfusate, the combined expression of the general white blood cell marker CD45 with specific membrane markers was used to identify each of the three immune cell types (FIG. 3C). In addition to using CD45, we identified Kupffer cell by CD14 surface antigen expression³⁰⁻³², Pit cells by expression of the general natural killer cell marker NKRP1A and the absence of CD3^{33,34}, and dendritic cell by the expression of OX62 and the absence of CD3³⁵. In this context, the presumed Kupffer, pit, and dendritic cells are referred to as CD45+/CD14+, CD45+/NKRP1A+/CD3−, and CD45+/OX62+/CD3− cells, respectively.

In contrast to the trend observed in structural liver cells, the percentage of Kupffer^{p} cells in the perfusate of fresh livers was high, but decreased with increasing durations of CI (FIG. 3A). We observed a sharp decline in Kupffer cell release in the 24-h-CI (0.78±0.39%) and 72-h-CI (0.42±0.33%) livers compared with fresh livers (4.07±0.60%; p<0.0001 and p<0.0001, respectively). Both the percentage (FIG. 3A) and absolute number (Table 3) of Kupffer cells in the 24-h-CI and 72-h-CI groups decreased by approximately two- and three-folds, respectively, compared with the fresh liver group; however, this result did not reach statistical significance.

Pitcells accounted for the largest population of released liver-resident immune cells and showed similar percentages for all experimental groups (16.66±1.95%, 15.74±10.19%, and 13.28±8.22% for fresh, 24-h-CI, and 72-h-CI livers, respectively). Dendritic cells are a rare cell type in the liver, which was consistent with the low percentages of dendritic cells (FIG. 3A) that were released into the perfusate (0.14±0.07%, 0.08±0.04%, and 0.08±0.04 for fresh, 24-h-CI, and 72-h-CI livers, respectively). No statistical differences were found between the liver groups for dendriticp cells.

Significant alterations in the liver-resident immune cell profiles during perfusion were observed following CI. Although these alterations did not significantly differentiate between moderately and severely injured CI livers, they could differentiate between fresh and CI livers.

### Example 4. Differences Between the Release of Structural Liver Cells at the Start and End of Perfusion

After analysis of the cells that were released in the perfusate that recirculated during perfusion, we now present and compare the release of liver specific cells derived from separate perfusion fractions that were flushed through the livers at the start of perfusion and at the very end of perfusion, as shown in the research design in FIGS. 1A-C.

No hepatocytes were released at the start of perfusion in fresh livers (0.11±0.06%), whereas after 24 h of CI, a significantly higher percentage of the released nucleated cells were hepatocytes (39.19±6.65%; p<0.0001) (FIG. 4A). Interestingly, the percentage of hepatocytesat the start of perfusion in 72-h-CI livers (4.79±4.36%) was significantly lower than that in 24-h-CI livers (p<0.0001). Additionally, the percentage of hepatocytes^{p} was higher at the start than at the end of perfusion in the 24-h-CI group (39.19±6.65% vs. 10.54±5.08%; p<0.0001), and the opposite trend was observed in the 72-h-CI group (4.79±4.36% vs. 26.93±7.24%; p<0.0001) (FIG. 4A). This may be caused by the lysis of injured hepatocytes after 72 h of CI or may also indicate delayed onset of hepatocyte detachment during perfusion as a function of ischemic injury.

Almost no LSECs^{p} were released at the beginning of perfusion in both the fresh controls and the 24-h- and 72-h-CI groups (0.02±0.02%, 0.12±0.10%, and 0.80±0.30%, respectively) (FIG. 3B). The LSEC percentages at the beginning of perfusion were very low and were not significantly different between the three liver groups. Almost no LSECs were released at the end of perfusion in fresh livers (0.05±0.07%). Interestingly, LSEC^{p} percentages in both the 24-h- and 72-h-CI livers were significantly higher at the end than at the start of perfusion (21.32±12.29%; p=0.0004 and 25.11±11.32%; p<0.0001, respectively).

The percentages of stellate cells in the perfusate were low at the start (0.15±0.10%, 1.58±0.83% and 2.43±1.10% for fresh, 24-h-CI, and 72-h-CI livers, respectively) and slightly higher at the end (0.56±0.78%, 1.84±0.99 and 3.03±0.92% for fresh, 24-h-CI, and 72-h-CI livers, respectively) of perfusion (FIG. 4C). However, the differences between the groups did not reach statistical significance.

### Example 5. Release of Liver-Resident Immune Cells at the Start and End of Perfusion

We hypothesized that the release of liver-resident immune cells, unlike that of structural liver cells, would be high at the start and lower at the end of perfusion due to a ‘wash-out’ effect during perfusion. This was confirmed by the significantly higher percentage of Kupffer cells at the beginning than at the end of perfusion in fresh, 24-h-CI, and 72-h-CI liver perfusates (5.99±0.70% vs. 3.89±0.48%, p<0.0001; 1.74±0.77% vs. 0.85±0.52%, p=0.0300; and 1.80±0.45% vs. 0.26±0.16%, p=0.0009, respectively) (FIG. 3D). Kupffer^{p} cell percentages in the perfusates flushed at the start of perfusion significantly decreased with increasing duration of ischemia (p<0.0001 for both fresh vs. 24-h-CI and fresh vs. 72-h-CI livers), which was similar to the perfusate that was collected during perfusion (FIG. 3A). The difference between the release of pit cells at the start and end of perfusion from cold ischemic livers showed an opposite trend to that of Kupffer cells (FIG. 4D). For both the 24-h-CI and 72-h-CI grafts, the percentage of pit cells was significantly lower at the start than at the end of perfusion (8.53±7.60% vs. 28.60±12.85%, p<0.0001 and 12.58±4.42% vs. 26.03±9.02%, p=0.0027, respectively) (FIG. 4E).

The percentage of dendritic cells released at the start of perfusion was significantly higher for the 72-h-CI group (0.43±0.34%) compared with that of both the fresh (0.14±0.06%) and the 24-h-CI (0.17±0.08%) groups (p=0.0072 and p=0.0089, respectively). The percentage of dendritic cell release at the end of perfusion was the same regardless ischemic injury (FIG. 4f).

### Example 6. Liver Function and Injury During Machine Perfusion as Result of Cold Ischemia

One important benefit of machine perfusion is that it allows the ex vivo assessment of liver viability. Although some of these parameters have been clinically correlated to transplant outcomes after normothermic machine perfusion, this remains to be determined for SNMP^{7,8,20,36,37}. Nonetheless, the different biochemical and mechanical parameters have strong theoretical background and are informative of liver function and injury during SNMP.

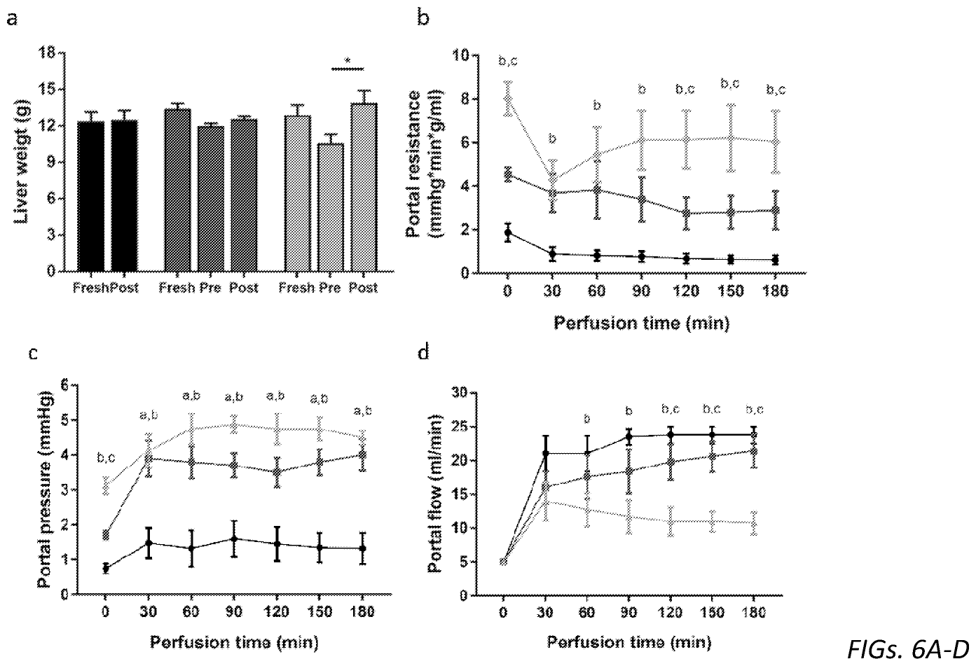

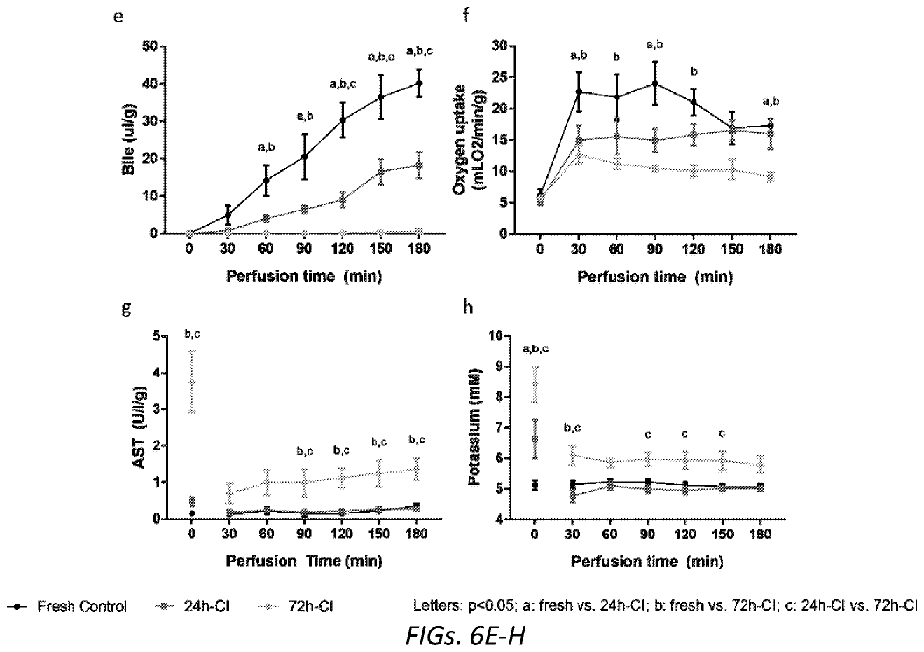

Liver weight is an indirect measure of cellular edema and we did not find significant differences in liver weights between the three groups (FIG. 6A). Microcirculatory dysfunction due to endothelial and stellate cell injuries, can cause increased vascular resistance³⁸. The vascular resistance (FIG. 6B) of the 72-h-CI livers was significantly higher than both the fresh (p<0.0339 at all time points) and the 24-h-CI (p<0.0385 at t=0; 120; 150; 180 min) livers. Likewise, fresh livers had significantly lower perfusion pressures (FIG. 6C) than the 24-h-CI livers (p<0.0015 at all time point after t=0 min) and 72-h-CI livers (p<0.0001 at all time points), while the flow (FIG. 6D) was significantly lower in the 72-h-CI livers as comparted to the fresh (p<0.0347 at all time points after t=30 min) and the 24-h-CI livers (p<0.0152 at all time points after t=90 min). Together this is indicative of significant endothelial injury in the 72-h-CI livers, which was consistent with our previous findings which correlate vascular resistance and transplant survival of rat livers after SNMP^{20,21}.

Both bile production and oxygen uptake are (hepato)cellular metabolic activity parameters which—despite the reduced metabolic rate at 21° C.—are normally observed during SNMP^{20,21,39-41}. The cumulative bile production (FIG. 6E) was at the highest levels in fresh (41.63±5.88 μl g⁻¹), at intermediate levels in the 24-h-CI livers (18.26±7.99), and was nearly absent in the 72-h-CI (0.62±0.13) livers (p<0.0001 at the end of perfusion between all groups). The oxygen uptake (FIG. 6F) followed the same trend, albeit significant differences in total oxygen uptake (area under the curve, AUC) was only observed between the fresh (3.55±0.70 l/g) and 72-h-CI livers (1.87±0.20; p=0.0061, Kruskal-Wallis test on AUC followed by the Dunn's post-hoc test).

Besides liver function and metabolic metrics such as bile production and oxygen uptake, we also assessed important parameters of liver injury in the venous outflow during perfusion.⁷. Whereas release of AST indicates hepatocyte-specific injury (FIG. 6G), potassium is a general parameter of cellular injury (FIG. 6H). Both the AST and potassium levels were highest in the perfusate of the 72-h-CI livers. The levels were the highest at the very start of perfusion (t=0 min), and directly dropped at t=30 min, due to a feature of the experimental design whereby the liver is flushed with 100 ml of perfusate to collect cells for imaging flow cytometry (i.e. the “start of perfusion” fraction, see schematic design in FIG. 1a). Whereas the AST activity of fresh and 24-h-CI livers remained similar and stable during perfusion, the AST activity of the 72-h-CI livers increased over time, indicative of ongoing hepatocellular injury during perfusion, which resulted in significantly higher values (1.38±0.59 U⁻¹ g⁻¹) compared with the fresh (0.35±0.17) and the 24-h-CI (0.28±0.08) livers at the end of perfusion (p=0.0109 and p=0.0036, respectively at t=180 min). The potassium levels also peaked at the start of perfusion in the 72-h-CI livers (8.43±1.15 mM). However, unlike AST, potassium levels were also elevated in the 24-h-CI livers (6.62±1.40) compared with the fresh livers (5.13±0.30; p<0.0003 between all groups at t=0 min).

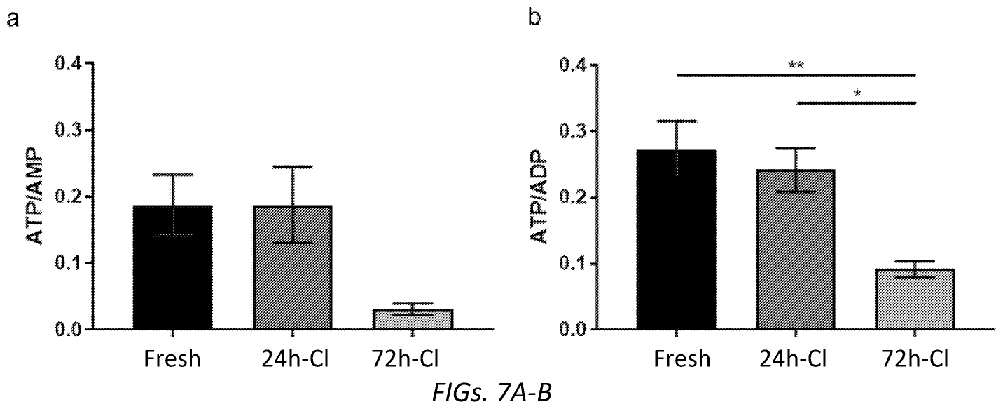

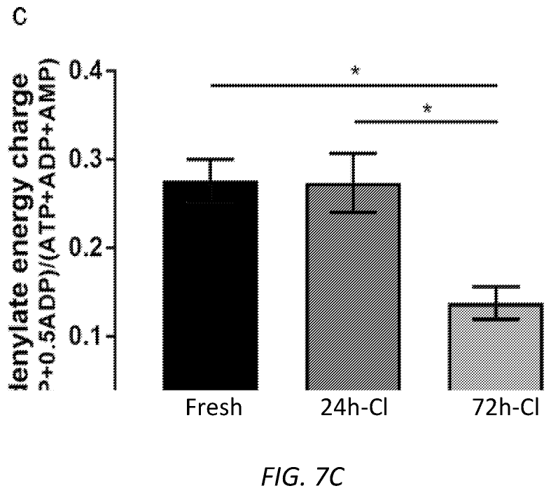

At the end of perfusion, we analyzed the energy status of the liver tissue based on the ratio of adenosine triphosphate (ATP), adenosine biphosphate (ADP), and adenosine monophosphate (AMP) (FIG. 7A-C). This is known as the adenylate energy charge which has been correlated to transplant survival in the used animal model²⁰ as well as to graft function after clinical liver transplantation^{44,45}. At the end of perfusion, the energy charge of fresh (0.28±0.05) and 24-h-CI livers (0.27±0.07) was significantly higher than the energy charge of 72-h-CI livers (0.14±0.04; p<0.0170 and p<0.0142, respectively), which agrees with our previous findings²⁰.

### Example 7. Morphologic Analysis of Cold Liver Ischemic Livers after Perfusion

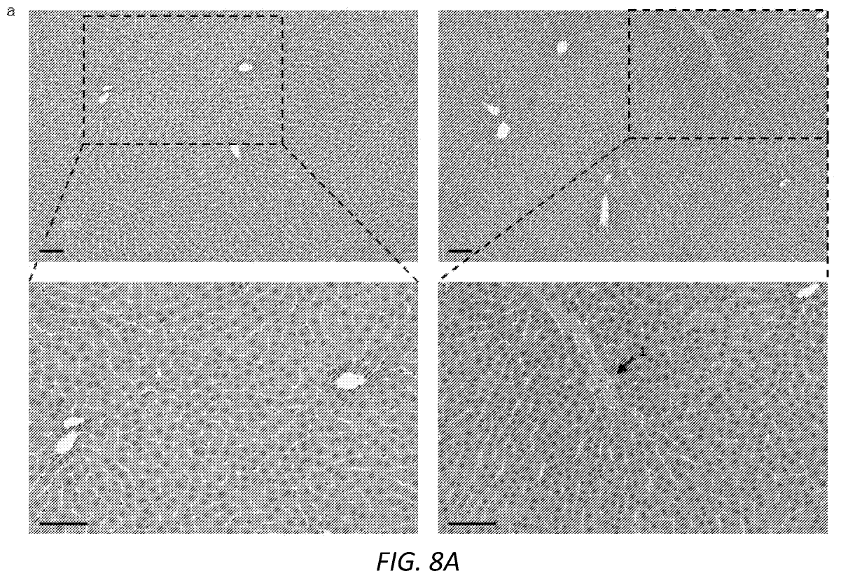

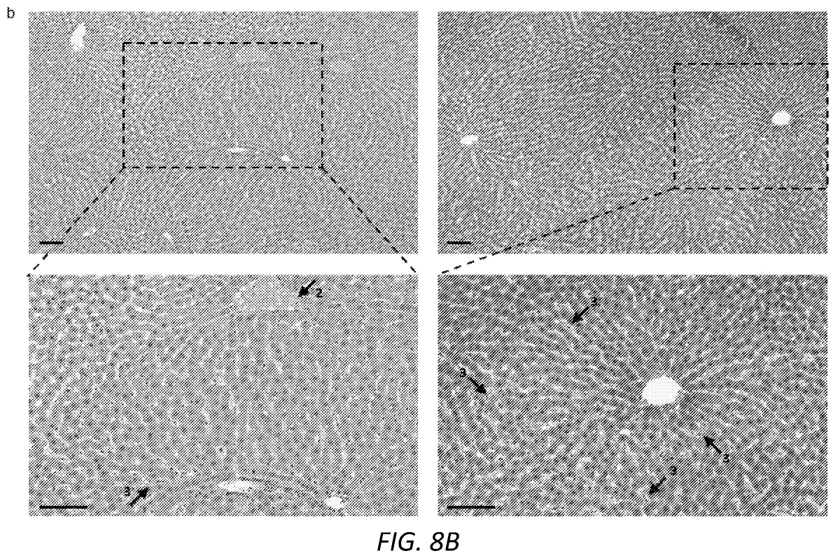

Additionally, we performed histological analysis of the liver tissue at the end of perfusion (FIG. 8A-B). Fresh controls showed normal lobular architecture without signs of injury. Liver lobules of the 24-h-CI livers showed well preserved architecture with mostly patent sinusoids and conspicuous LSECs. Mild congestion of liver sinusoids and focal disruption of endothelial lining with detached cells in central veins and cellular debris in the lumen was seen. Hepatocytes showed mild reactive changes in the form of scattered binucleated hepatocytes and hydropic degeneration with scarce hepatocyte dropout. The 72-h-CI livers showed preserved lobular architecture, although congestion of the sinusoids was evident. Focal disruption of endothelial lining was present and detached cells, eosinophilic granular and cellular debris were seen the lumen of both portal and central veins. LSECs appeared loosely attached and displaced by perisinusoidal subendothelial edema in the space of Disse. Reactive hepatocytes changes and parenchymal drop out with patchy centrilobular hydropic degeneration were more present as compared to 24-h-CI livers.

### Example 8. Correlations Between Cell Release and Biochemical Parameters of Liver Function and Injury

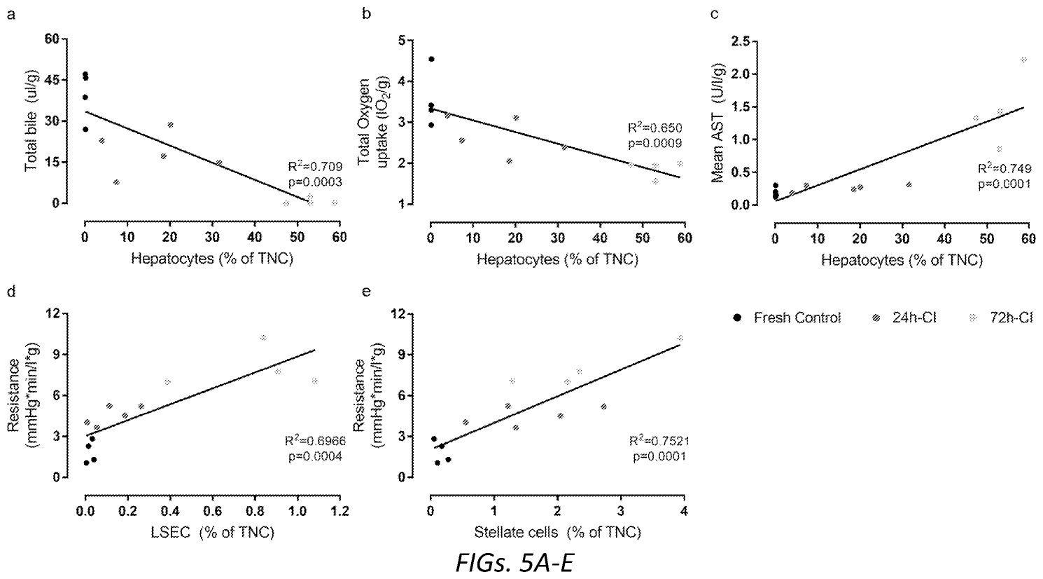

Because ischemia leads to hepatocyte injury and hepatocytes are solely responsible for bile excretion, we hypothesized that bile production could be related to hepatocyte release. We found a significant negative correlation between total bile production and the percentage of hepatocytes in the perfusate (p=0.0003, R²=0.709; linear regression throughout the text) (FIG. 5A). Because 80% of the liver cells are hepatocytes, they represent the bulk of oxygen uptake measured for the whole liver. Therefore, we also tested the relationship between total oxygen uptake (AUC) and hepatocyte release during perfusion and found a significant negative correlation (p=0.0009, R²=0.650) between them (FIG. 5B). Similarly, we found a significant positive correlation between AST levels and hepatocyte release (p=0.0001, R²=0.749) (FIG. 5C).

Whereas hepatocyte release during perfusion was most strongly related to the degree of ischemic injury during HP, LSEC and stellate cell releases were more indicative of cold ischemic injury in the perfusate fractions collected at the very start of perfusion (FIGS. 4A-F). LSEC and stellate cells play a crucial role in liver microcirculation^{38,46}, which is most reflected by vascular resistance during perfusion. Therefore, we expected a positive correlation between LSEC and stellate cell release with vascular resistance at the start of perfusion. Indeed, the release of both LSEC and stellate cells in the perfusate fractions collected at the start of perfusion were strongly and significantly correlated to vascular resistance at the start of perfusion (p=0.0004, R2=0.6966 and p=0.0001, R²=0.7521, respectively) (FIGS. 5D and 5E, respectively).

### Example 9. Post-Transplant Monitoring—Imaging Flow Cytometry for the Identification of Liver-Derived Donor Cells

This example describes methods of measuring the immunosuppressive state of the graft after transplantation into a recipient. Imaging flow cytometry panels with the capacity to identify hepatocytes (HC), liver sinusoidal endothelial cells (LSEC), Kupffer cells (KC), hepatic stellate cells (SC), dendritic cells (DC), while also differentiating against peripheral cells are used. An ImageStreamx Mark II imaging flow cytometer (Amnis Corporation) is used, equipped with a 40× objective, 6 imaging channels, and 405 nm, 488 nm, and 642 nm lasers. Markers outlined in Table B are used for identification of each of liver cell types. We validate cross reactivity of each antibody using established cell lines known to express each surface marker. At the same time, negative controls consist of cell lines known for the absence of the antigen of interest. After initial antibody validation, we use isolated human livers cells. Using each of these cell fractions, antibody concentration and duration of staining are tested against the laser power to ensure adequate fluorescence without saturation. Analysis of cells derived from human liver without blood components is used to identify expression signatures and enable us to design expected cell gates. Finally, liver cells are spiked into whole blood and gating and antibody specificity is confirmed by flow cytometry. The imaging flow cytometry panel is used for accurate identification of liver derived cells while also excluding cells originating from peripheral blood.

### Example 10. Clinical Samples to Identify Populations of Rare Circulating Liver-Derived Donor Cells

This example describes isolation of liver derived cells from peripheral blood from transplant recipients. To achieve this, liver transplant patients who have been diagnosed with acute rejection are enrolled. Patients are enrolled when rejection has been confirmed with a tissue biopsy and liver function tests (LFTs) are twice normal. Such biopsies and LFTs are standard of care and thus additional tests for the purpose of this study are not required. In addition to the experimental cohort of transplant patients with elevated LFTs, we also include control groups to look at different types of injury that may not be immune related and which can also have rising LFTs, for example, alcohol/drug hepatitis and fatty liver disease. We process transplant recipients without rejection (defined as normal LFTs) and healthy controls.

Peripheral blood is sampled immediately after confirmed diagnosis of rejection and patients are tracked throughout their treatment course with additional blood sampled after LFTs have returned to normal and 6 months after the acute rejection episode has resolved. Blood samples are obtained and processed, e.g., within <6 hours of blood draw. Using cell sorting technology to find rare cells and a flow cytometry assay, we classify the types of liver cells present in transplant patient peripheral blood and quantify the concentration of each liver cell type per mL of blood. At the same time, patient biopsies and charts are reviewed for degree of endothelitis (classified as lifting of the endothelium (mild), disruption of the intact endothelial lining (medium), and damage to adjacent hepatocytes (severe)), levels of LFTs, complications, other diagnostic studies, and graft/patient survival by study staff that is blinded to the results of the cell analysis. Medical record review includes demographics and include: age, sex, gender, date of transplant, reason for transplant, type of transplant (e.g. living donor, deceased donor), type of immunosuppression, laboratory values, patient hospitalization duration, cold ischemia and warm ischemia time, biliary complications and procedures, and biopsy results.

Using the concentration of each liver cell type per mL of blood and patient information, as described above, we perform a regression analysis to evaluate the correlation between our potential cell biomarkers and graft rejection, defined as confirmed endothelitis in tissue biopsies and LFTs twice normal. As a secondary endpoint we perform a regression analysis to evaluate the correlation between cell biomarkers and allograft dysfunction, defined as ALT>2000 U.

### Example 11. Detection of Renal Rejection

To determine the immunosuppressive state of a renal graft, we sample peripheral blood to isolate and characterize cells derived from the graft.

Human kidney cells. Kidneys are complex organs that perform many functions. In addition to filtering blood, they secrete hormones that regulate blood pressure and maturation of red blood cells. Many of the filtration functions also have secondary effects, such as maintaining bone health and controlling the acid-base balance in the body. Given these numerous functions, it is not surprising that kidneys are made up of many cell types. Thus, our isolation technology isolates diverse kidney cell types. Initial testing and optimization is performed with diverse human kidney cell lines obtained from commercial vendors. This includes representative kidney cells that make up the glomerulus (podocytes, mesangial cells), Bowman's capsule (parietal cells, juxtaglomerulal cells), and tubules and the loop of Henle (proximal tubule, thick ascending limb, and distal tubule cells).

Blood collection & microfluidic isolation of kidney-derived cells. Fresh healthy donor blood is collected in ACD-A anticoagulant tube from our in-house donor pool of over 50 healthy members, as well as purchased externally from reputable organizations. For spiked cell experiments (2-1,000 cells/mL blood), kidney-derived cells are added directly to the blood collection tube to serve as a model. For microfluidic isolation of spiked cells and enumeration experiments, CellTracker (Life Technologies)-labeled human kidney cells are resuspended in PBS and spiked into healthy donor blood at various cell concentrations per milliliter of blood (note: transplant patient blood specimens are used without spiking). We use ACD vacutainers which are the preferred blood collection tube for microfluidic isolation and preservation of circulating tumor cells (CTCs) from peripheral blood (Wong et al. Nat. Commun. 8, 1733 (2017)).

Spiked blood samples are incubated for 15 minutes prior to incubation with biotinylated depletion antibodies to the CD45 antigen on white blood cells followed by a second incubation with streptavidin-coupled Dynabeads (Invitrogen), and loaded into a pressurized syringe for microfluidic processing, as described below. This method first separates nucleated cells from other blood components by removing plasma, platelets, RBCs, and free beads. Two debulking methods are tested, including deterministic lateral displacement vs. a non-equilibrium inertial separation array (NISA) (Mutlu et al., Sci. Rep. 7, 9915 (2017)). Following debulking, nucleated cells are aligned in a single file by inertial focusing and magnetically deflecting bead-labeled leukocytes under continuous flow; see FIG. 10. Highly enriched kidney cells are collected in the product outlet in PBS containing 1% (w/v) Pluronic F-68 (Sigma). Spiked cells are enumerated by counting CellTracker-positive cells in two Nageotte chambers (Hausser Scientific). Leukocytes are enumerated as nucleated cells that were not spiked cells (positive for DyeCycle green, negative for Cell Tracker red).

Blood is obtained from kidney transplant recipients. All samples are processed within 6 hours of blood draw, and isolated circulating kidney cells are enumerated and characterized by imaging flow cytometry and digital droplet PCR. Prior to microfluidic processing, we add our platelet inhibitor cocktail, as previously described (Wong et al. Nat. Commun. 8, 1733 (2017)). The sample is then processed using the iChip to isolate circulating kidney cells. This is followed by enumeration and molecular assays to determine the origin of the kidney cells via HLA markers (i.e. either from donor or recipient).

Imaging flow cytometry panels with the capacity to identify endothelial cells, mesangial cells, podocytes, renal pariental cells, juxtaglomerulal cells, proximal tubule cells, thick ascending limb cells, and distal tubule cells, while also differentiating against peripheral white blood cells (WBC), other vascular endothelial cells (VEC), platelets, and hemopoietic stem cells are used. Our ImageStreamx Mark II imaging flow cytometer (Amnis Corporation) is equipped with a 40× objective, 6 imaging channels, and 405 nm, 488 nm, and 642 nm lasers. Markers outlined in Table C are used for identification of each renal-specific cell type. We validate cross reactivity of each antibody using established cell lines known to express each surface marker. At the same time, negative controls consist of cell lines known for the absence of the antigen of interest. After initial antibody validation, we use isolated human renal cells. Using each of these cell fractions, antibody concentration and duration of staining are tested against the laser power to ensure adequate fluorescence without saturation. Analysis of cells derived from human kidney without blood components is used to identify expression signatures and enable us to design expected cell gates. Finally, kidney cells are spiked into whole blood and gating and antibody specificity is confirmed by flow cytometry. The imaging flow cytometry panel is used for accurate identification of kidney derived cells while also excluding cells originating from peripheral blood.

### Example 12. Clinical Samples to Identify Populations of Rare Circulating Kidney-Derived Donor Cells

This example describes isolation of kidney derived cells from peripheral blood from transplant recipients. Patients are enrolled (˜50 patients, with up to 15 of these patients expected to be diagnosed with acute rejection), and blood is sampled at the following time points post-transplantation: twice in the first week, once/week for three weeks, once/month for several months. Depending on the enrolment date of the patient, this would result in 8-16 blood draw per patient. When possible, we also collect samples pre-transplant to act as the baseline control. Samples are obtained and processed within <6 hours of blood draw and with best practices as determined in previous objectives. Using an imaging flow cytometry protocol, as described above, we classify the types of kidney cells present in transplant patient peripheral blood and quantify the concentration of each kidney cell type per mL of blood. Further, we use digital droplet PCR against specific HLA markers to quantify the ratio of kidney cells present in the product originating from the donor versus recipient.

### REFERENCES

1. Asrani, S. K., Devarbhavi, H., Eaton, J. & Kamath, P. S. Burden of liver diseases in the world. J. Hepatol. 70, 151-171 (2019).
2. Kim, W. R. et al. OPTN/SRTR 2017 Annual Data Report: Liver. Am.

J. Transplant. 19, 184-283 (2019).
3. Giwa, S. et al. The promise of organ and tissue preservation to transform medicine. Nat. Biotechnol. 35, 530-542 (2017).
4. Abt, P. L. et al. Survival following liver transplantation from non-heart-beating donors. Ann. Surg. 239, 87-92 (2004).
5. Feng, S. et al. Characteristics Associated with Liver Graft Failure: The Concept of a Donor Risk Index. Am. J. Transplant. 6, 783-790 (2006).
6. Flores, A. & Asrani, S. K. The donor risk index: A decade of experience. Liver Transpl. 23, 1216-1225 (2017).
7. Watson, C. J. E. & Jochmans, I. From ‘Gut Feeling’ to Objectivity: Machine Preservation of the Liver as a Tool to Assess Organ Viability. Curr.

Transplant. Rep. 5, 72-81 (2018).
8. Ceresa, C. D. L., Nasralla, D. & Jassem, W. Normothermic Machine Preservation of the Liver: State of the Art. Curr. Transplant. Rep. 5, 104-110 (2018).
9. Dutkowski, P. et al. Evolving Trends in Machine Perfusion for Liver Transplantation. Gastroenterology (2019) doi:10.1053/j.gastro.2018.12.037.
10. Marecki, H. et al. Liver ex situ machine perfusion preservation: A review of the methodology and results of large animal studies and clinical trials. Liver Transpl. 23, 679-695 (2017).
11. Perera, T. et al. First human liver transplantation using a marginal allograft resuscitated by normothermic machine perfusion. Liver Transpl. 22, 120-124 (2016).
12. Krishna, M. Microscopic anatomy of the liver. Clin. Liver Dis. 2, S4-S7(2013).
13. Fan, X. & Rudensky, A. Y. Hallmarks of Tissue-Resident Lymphocytes. Cell 164, 1198-1211 (2016).
14. Jenne, C. N. & Kubes, P. Immune surveillance by the liver. Nat.

Immunol. 14, 996-1006 (2013).
15. Nakatani, K., Kaneda, K., Seki, S. & Nakajima, Y. Pit cells as liver-associated natural killer cells: morphology and function. Med. Electron Microsc. Off.

J. Clin. Electron Microsc. Soc. Jpn. 37, 29-36 (2004).
16. Eltzschig, H. K. & Eckle, T. Ischemia and reperfusion—from mechanism to translation. Nat. Med. 17, (2011).
17. Wallach, D., Kang, T.-B. & Kovalenko, A. Concepts of tissue injury and cell death in inflammation: a historical perspective. Nat. Rev. Immunol. 14, 51-59 (2014).
18. van Golen, R. F., van Gulik, T. M. & Heger, M. The sterile immune response during hepatic ischemia/reperfusion. Cytokine Growth Factor Rev. 23, 69-84 (2012).
19. Stewart, Z. A. UW Solution: Still the ‘Gold Standard’ for Liver Transplantation. Am. J. Transplant. 15, 295-296 (2015).
20. Bruinsma, B. G., Berendsen, T. A., Izamis, M.-L., Yarmush, M. L. & Uygun, K. Determination and extension of the limits to static cold storage using subnormothermic machine perfusion. Int. J. Artif Organs 36, 775-780 (2013).
21. Berendsen, T. A. et al. Supercooling enables long-term transplantation survival following 4 days of liver preservation. Nat. Med. 20, 790-793 (2014).
22. Willoughby, J. L. S. et al. Evaluation of GalNAc-siRNA Conjugate Activity in Pre-clinical Animal Models with Reduced Asialoglycoprotein Receptor Expression. Mol. Ther. 26, 105-114 (2018).
23. Hrzenjak, A. et al. Galactose-specific asialoglycoprotein receptor is involved in lipoprotein (a) catabolism. Biochem. J. 376, 765-771 (2003).
24. Tissue expression of ASGR1—Staining in liver—The Human Protein Atlas. proteinatlas.org/ENSG00000141505-ASGR1/tissue/liver.
25. March, S., Hui, E. E., Underhill, G. H., Khetani, S. & Bhatia, S. N. Microenvironmental regulation of the sinusoidal endothelial cell phenotype in vitro. Hepatol. Baltim. Md. 50, 920-928 (2009).
26. Bale, S. S. et al. Long-Term Coculture Strategies for Primary Hepatocytes and Liver Sinusoidal Endothelial Cells. Tissue Eng. Part C Methods 21, 413-422 (2015).
27. Meurer, S. K., Tihaa, L., Lahme, B., Gressner, A. M. & Weiskirchen, R. Identification of Endoglin in Rat Hepatic Stellate Cells NEW INSIGHTS INTO TRANSFORMING GROWTH FACTOR 3 RECEPTOR SIGNALING. J. Biol. Chem. 280, 3078-3087 (2005).
28. Strauss, O., Phillips, A., Ruggiero, K., Bartlett, A. & Dunbar, P. R. Immunofluorescence identifies distinct subsets of endothelial cells in the human liver. Sci. Rep. 7, 44356 (2017).
29. Connolly, M. K. et al. In Hepatic Fibrosis, Liver Sinusoidal Endothelial Cells Acquire Enhanced Immunogenicity. J. Immunol. Baltim. Md. 1950 185, 2200-2208 (2010).
30. Qiu, D. K., Hua, J., Li, J. Q. & Li, E. L. CD14 expression on Kupffer cells during the course of carbon tetrachloride-mediated liver injury. Chin. J. Dig. Dis. 6, 137-141 (2005).
31. Dai, L.-L. et al. Synthesis of endotoxin receptor CD14 protein in Kupffer cells and its role in alcohol-induced liver disease. World J. Gastroenterol. 9, 622-626 (2003).
32. Su, G. L. et al. Activation of human and mouse Kupffer cells by lipopolysaccharide is mediated by CD14. Am. J. Physiol. Gastrointest. Liver Physiol. 283, G640-645 (2002).
33. Gao, B., Radaeva, S. & Park, O. Liver natural killer and natural killer T cells: immunobiology and emerging roles in liver diseases. J. Leukoc. Biol. 86, 513-528 (2009).
34. Peng, H., Wisse, E. & Tian, Z. Liver natural killer cells: subsets and roles in liver immunity. Cell. Mol. Immunol. 13, 328-336 (2016).
35. Lau, A. H. & Thomson, A. W. Dendritic cells and immune regulation in the liver. Gut 52, 307-314 (2003).
36. Sutton, M. E. et al. Criteria for viability assessment of discarded human donor livers during ex vivo normothermic machine perfusion. PloS One 9, e110642 (2014).
37. Perk, S. et al. A metabolic index of ischemic injury for perfusion-recovery of cadaveric rat livers. PloS One 6, e28518 (2011).
38. Jain, S. et al. Ex-vivo study of flow dynamics and endothelial cell structure during extended hypothermic machine perfusion preservation of livers.

Cryobiology 48, 322-332 (2004).
39. Berendsen, T. A. et al. A simplified subnormothermic machine perfusion system restores ischemically damaged liver grafts in a rat model of orthotopic liver transplantation. Transplant. Res. 1, 6 (2012).
40. Bruinsma, B. G. et al. Subnormothermic machine perfusion for ex vivo preservation and recovery of the human liver for transplantation. Am. J. Transplant. Off. J. Am. Soc. Transplant. Am. Soc. Transpl. Surg. 14, 1400-1409 (2014).
41. Bruinsma, B. G. et al. Metabolic profiling during ex vivo machine perfusion of the human liver. Sci. Rep. 6, (2016).
42. Bruinsma, B. G. et al. Metabolic profiling during ex vivo machine perfusion of the human liver. Sci. Rep. 6, 22415 (2016).
43. Bruinsma, B. G. et al. Subnormothermic Machine Perfusion for Ex Vivo Preservation and Recovery of the Human Liver for Transplantation: Subnormothermic Machine Perfusion of Human Livers. Am. J. Transplant. 14, 1400-1409 (2014).
44. Bruinsma, B. G. et al. Peritransplant Energy Changes and Their Correlation to Outcome After Human Liver Transplantation: Transplantation 101, 1637-1644 (2017).
45. Vajdovi, K., Graf, R. & Clavien, P.-A. ATP-supplies in the cold-preserved liver: A long-neglected factor of organ viability. Hepatol. Baltim. Md. 36, 1543-1552 (2002).
46. Reynaert, H., Thompson, M. G., Thomas, T. & Geerts, A. Hepatic stellate cells: role in microcirculation and pathophysiology of portal hypertension. Gut 50, 571-581 (2002).
47. Erdbruegger, U., Dhaygude, A., Haubitz, M. & Woywodt, A.

Circulating endothelial cells: markers and mediators of vascular damage. Curr. Stem Cell Res. Ther. 5, 294-302 (2010).
48. Molina, D. K. & DiMaio, V. J. M. Normal organ weights in men: part II—the brain, lungs, liver, spleen, and kidneys. Am. J. Forensic Med. Pathol. 33, 368-372 (2012).
49. Kalogeris, T., Baines, C. P., Krenz, M. & Korthuis, R. J. Cell Biology of Ischemia/Reperfusion Injury. Int. Rev. Cell Mol. Biol. 298, 229-317 (2012).
50. Sohlenius-Sternbeck, A.-K. Determination of the hepatocellularity number for human, dog, rabbit, rat and mouse livers from protein concentration measurements. Toxicol. Vitro Int. J. Publ. Assoc. BIBRA 20, 1582-1586 (2006).
51. Clavien, P.-A. Sinusoidal endothelial cell injury during hepatic preservation and reperfusion. Hepatology 28, 281-285 (1998).
52. Stolz, D. B. et al. Sinusoidal endothelial cell repopulation following ischemia/reperfusion injury in rat liver transplantation. Hepatology 46, 1464-1475 (2007).
53. Miyashita, T. et al. Ischemia reperfusion-facilitated sinusoidal endothelial cell injury in liver transplantation and the resulting impact of extravasated platelet aggregation. Eur. Surg. 48, 92-98 (2016).
54. Jameel, N. M. et al. Constitutive release of powerful antioxidant-scavenging activity by hepatic stellate cells: Protection of hepatocytes from ischemia/reperfusion injury. Liver Transpl. 16, 1400-1409 (2010).
55. Stewart, R. K. et al. A novel mouse model of depletion of stellate cells clarifies their role in ischemia/reperfusion- and endotoxin-induced acute liver injury.

J. Hepatol. 60, 298-305 (2014).
56. Zhang, C.-Y., Yuan, W.-G., He, P., Lei, J.-H. & Wang, C.-X. Liver fibrosis and hepatic stellate cells: Etiology, pathological hallmarks and therapeutic targets. World J. Gastroenterol. 22, 10512-10522 (2016).
57. Magee, N., Zou, A. & Zhang, Y. Pathogenesis of Nonalcoholic Steatohepatitis: Interactions between Liver Parenchymal and Nonparenchymal Cells.

BioMed Res. Int. 2016, (2016).
58. Tacke, F. New Insights on the Role of Kupffer Cells in Liver Transplantation. Transplantation 102, 896 (2018).
59. Verhoeven, C. J. et al. Biomarkers to assess graft quality during conventional and machine preservation in liver transplantation. J. Hepatol. 61, 672-684 (2014).
60. Henry, S. D. et al. Hypothermic machine preservation reduces molecular markers of ischemia/reperfusion injury in human liver transplantation. Am. J. Transplant. Off. J. Am. Soc. Transplant. Am. Soc. Transpl. Surg. 12, 2477-2486 (2012).
61. Sridharan, G. V. et al. Metabolomic Modularity Analysis (MMA) to Quantify Human Liver Perfusion Dynamics. Metabolites 7, (2017).
62. Boteon, Y. L. & Afford, S. C. Machine perfusion of the liver: Which is the best technique to mitigate ischaemia-reperfusion injury? World J. Transplant. 9, 14-20 (2019).
63. Karabacak, N. M. et al. Microfluidic, marker-free isolation of circulating tumor cells from blood samples. Nat. Protoc. 9, 694-710 (2014).
64. Bhan, I. et al. Detection and Analysis of Circulating Epithelial Cells in Liquid Biopsies From Patients With Liver Disease. Gastroenterology 155, 2016-2018.e11 (2018).
65. Bale, S. S., Geerts, S., Jindal, R. & Yarmush, M. L. Isolation and coculture of rat parenchymal and non-parenchymal liver cells to evaluate cellular interactions and response. Sci. Rep. 6, 25329 (2016).
66. Dunn, J. C., Tompkins, R. G. & Yarmush, M. L. Long-term in vitro function of adult hepatocytes in a collagen sandwich configuration. Biotechnol. Prog. 7, 237-245 (1991).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining viability of an organ for transplantation during or after Machine Perfusion (MP), optionally Sub-Normothermic MP (SNMP), or preservation, optionally Hypothermic Preservation (HP), the method comprising:
providing a sample of perfusate from the MP or preservation storage media;
detecting whole cells from the organ in the sample, and determining the identity, level, activity and/or status, of selected cell types in the sample;
comparing the level of the cells of the selected type from the organ to a reference identity, level, activity and/or status of cells of the selected type, wherein the reference level represents a level, activity and/or status in a viable organ; and
identifying an organ that has a level, activity and/or status of cells of the selected type that differs from the reference identity, level, activity and/or status as unsuitable for transplant; or
identifying an organ that has a level, activity and/or status of cells of the selected type comparable to the reference identity, level, activity and/or status as suitable for transplant.

2. The method of claim 1, wherein an organ has been identified as having a level of cells of the selected type below the reference level as suitable for transplant, and the method further comprises preparing the organ for transplant and optionally transplanting the organ into a suitable recipient.

3. The method of claim 1, further comprising enriching the sample for cells from the organ.

4. The method of claim 1, wherein:
(a) the organ is a liver and the cells are hepatocytes (HC), liver sinusoidal endothelial cells (LSEC), Kupffer cells (KC), Pit Cells, hepatic stellate cells (SC), fibroblasts, and/or dendritic cells (DC),
(b) the organ is a kidney and the cells are endothelial cells, mesangial cells, podocytes, renal pariental cells, juxtaglomerulal cells, proximal tubule cells, thick ascending limb cells, and distal tubule cells;
(c) the organ is a heart and the cells are coronary endothelial cells, cardiomyocytes, cardiac pacemaker cells, and/or cardiac fibroblasts;
(d) the organ is a lung and the cells are, pneumocytes, alveolar macrophages, pulmonary dendritic cells, pulmonary fibroblasts, and/or pulmonary endothelial cells;
(e) the organ is a pancreas and the cells are alpha cells, beta cells, delta cells, pancreatic islet endothelial cells, pancreatic polypeptide cells, acinar cells, and/or pancreatic fibroblasts;
(f the organ is a vascularized composite allograft and the cells are rhabdomyocytes, Langerhans cells, keratinocytes, dermal dendritic cells, mast cells, lymphocytes, capillary endothelial cells, Schwann cells, osteoblasts, and/or osteoclasts; or
(g) the organ is a small intestine and the cells are Paneth cells, Goblet cells, enteroendocrine cells, enterocytes, intestinal dendritic cells, intestinal macrophages and/or gut endothelial cells.

5. (canceled)

6. (canceled)

7. (canceled)

8. (canceled)

9. (canceled)

10. (canceled)

11. The method of claim 1, wherein determining the identity, level, activity and/or status of selected cell types in the sample comprises contacting the sample with antibodies that bind to identifying cell surface antigens and or intracellular proteins on/in the selected cell types, and quantifying cells that are bound to the antibodies.

12. The method of claim 1, wherein determining the identity, level, activity, and/or status of selected cell types in the sample comprises quantification of DNA, RNA, protein, metabolites, methylation status or other post-translational modifications, or other biomolecule indicating identity, level, activity and/or status of the cells.

13. A method of detecting donor organ rejection or injury or risk of donor organ rejection or injury in a subject, the method comprising:
providing a sample comprising peripheral blood from the subject;
enriching the sample for donor organ-derived cells;
detecting whole cells from the organ in the sample, and determining the identity, level, activity and/or status of selected cell types in the sample;
comparing the identity, level, activity and/or status of the cells of the selected type from the organ to a reference identity, level, activity and/or status of cells of the selected type; and
identifying a subject who has a level of cells of the selected type above the reference level as having donor organ rejection or at risk of developing donor organ rejection or injury, and administering a treatment for rejection or injury or to reduce the risk of rejection or injury to the organ to the subject; or
identifying a subject who has a level of cells of the selected type below the reference level as not having donor organ rejection or injury or not at risk of donor organ rejection or injury.

14. The method of claim 13, further comprising enriching the sample for cells from the organ.

15. The method of claim 14, wherein enriching the sample for donor organ-derived cells comprises separating nucleated cells from other blood components; collecting and removing peripheral leukocytes; contacting the sample with antibodies that bind to identifying cell surface antigens or intracellular proteins in the selected cell types, and quantifying cells that are bound to the antibodies.

16. The method of claim 15, comprising using a microfluidic device for enriching the sample for donor organ-derived cells.

17. The method of claim 13, wherein determining the identity, level, activity and/or status of selected cell types in the sample comprises contacting the sample with antibodies that bind to identifying cell surface antigens on or intracellular proteins in the selected cell types, and quantifying cells that are bound to the antibodies.

18. The method of claim 13, wherein determining the identity, level, activity and/or status of selected cell types in the sample comprises quantification of DNA, RNA, protein, or other biomolecule indicating identity, level, activity and/or status of the cells.

19. The method of claim 13, wherein the organ is a liver and the cells are hepatocytes (HC), liver sinusoidal endothelial cells (LSEC), Kupffer cells (KC), hepatic stellate cells (SC), Pit cells, fibroblasts, and/or dendritic cells (DC).

20. The method of claim 13, wherein the organ is a kidney and the cells are endothelial cells, mesangial cells, podocytes, renal pariental cells, juxtaglomerulal cells, proximal tubule cells, thick ascending limb cells, and distal tubule cells.

21. The method of claim 13, wherein the organ is a heart and the cells are coronary endothelial cells, cariomyocytes, cardiac pacemaker cells, and/or cardiac fibroblasts.

22. The method of claim 13, wherein the organ is a lung and the cells are pneumocytes, alveolar macrophages, pulmonary dendritic cells, pulmonary fibroblasts, and/or pulmonary endothelial cells.

23. The method of claim 13, wherein the organ is a pancreas and the cells are alpha cells, beta cells, delta cells, pancreatic islet endothelial cells, pancreatic polypeptide cells, acinar cells, and/or pancreatic fibroblasts.

24. The method of claim 13, wherein the organ is a vascularized composite allograft and the cells are rhabdomyocytes, Langerhans cells, keratinocytes, dermal dendritic cells, mast cells, lymphocytes, capillary endothelial cells, Schwann cells, osteoblasts, and/or osteoclasts.

25. The method of claim 13, wherein the organ is a small intestine and the cells are Paneth cells, Goblet cells, enteroendocrine cells, enterocytes, intestinal dendritic cells, intestinal macrophages and/or gut endothelial cells.

26. The method of claim 13, wherein determining the identity, level, activity and/or status of selected cell types in the sample comprises (optionally amplifying) and quantifying one or more cell-type specific transcripts or proteins from donor organ-derived cells, wherein the quantity of the cell-type specific transcripts or proteins indicates the identity, level, activity and/or status of the donor organ-derived cells.
